(19) United States
(12) Patent Application Publication
(10) Pub. No.: US 2005/0101640 A1
(43) Pub. Date: May 12, 2005

(54) Lisinopril/lercanidipine combination therapy

(75) Inventors: Amedeo Leonardi, Milan (IT); Abraham Sartani, Arese (IT); Giorgio Sironi, Pieve Emanuele (IT)

Correspondence Address: DARBY & DARBY P.C., P. O. BOX 5257, NEW YORK, NY, 10150-5257, US

(73) Assignee: Recordati Ireland Limited

(21) Appl. No.: 10/835,095

(22) Filed: Apr. 28, 2004

Related U.S. Application Data
(63) Continuation-in-part of application No. 10/688,061, filed on Oct. 16, 2003.
(60) Provisional application No. 60/439,884, filed on Jan. 14, 2003.
(60) Provisional application No. 60/419,790, filed on Oct. 16, 2002.

(30) Foreign Application Priority Data
Dec. 6, 2002 (IT) MI 2002 A 002594

(51) Int. Cl. A61K 31/455; A61K 31/401

(52) U.S. Cl. 514/355; 514/423

(57) ABSTRACT

Disclosed are compositions and methods for treating hypertension comprising lisinopril and lercanidipine and optionally including a diuretic in amounts effective in combination to reduce blood pressure to a patent in need of treatment.

### CROSS-REFERENCE TO RELATED APPLICATIONS

This application is a continuation-in-part of Ser. No. 10/688,061, filed Oct. 16, 2003 and claims the benefit of priority under 35 U.S.C. § 119(e) of provisional application Ser. No. 60/419,790, filed Oct. 16, 2002, and 60/439,884, filed Jan. 14, 2003, and the benefit of priority under 35 U.S.C. §§ 119(a)-(d) of Italian application MI 2002A 002594, filed Dec. 6, 2002. Each of the foregoing applications is hereby incorporated herein by reference in its entirety.

### FIELD OF THE INVENTION

The present invention contemplates a method for treating hypertension with a combination of lisinopril and lercanidipine, optionally further in combination with a diuretic, such as hydrochlorothiazide (HCTZ).

### BACKGROUND OF THE INVENTION

Hypertension is one of the most common cardiovascular disease states. In the United States, over 50 million people have been diagnosed with hypertension (which is defined as a blood pressure greater than or equal to 140/90 mm Hg). Elevated arterial pressure can cause pathological changes in the vasculature and hypertrophy of the left ventricle. Due to the damage that can be produced by hypertension, it is proposed to be the principal cause of stroke, myocardial infarction, and sudden cardiac death. Additionally, hypertension is believed to be a major contributor to cardiac failure, renal insufficiency, and dissecting aneurysm of the aorta.

The renin-angiotensin system is an important regulator of arterial pressure. The inactive angiotensinogen peptide is converted to the pro-peptide angiotensin I by the enzyme renin. Angiotensin I then is converted to the active angiotensin II form by the angiotensin converting enzyme (ACE). Angiotensin II then acts through a variety of receptor mediated mechanisms, such as increasing the total peripheral resistance and inhibiting the excretion of sodium and water by the kidneys, to increase arterial pressure.

ACE inhibitors are active agents that prevent the conversion of angiotensin I into angiotensin II. The hypotensive action of these active agents is well documented and such active agents have been used extensively in the treatment of hypertension. Examples of ACE inhibitors are described in U.S. Pat. Nos. 4,350,633; 4,344,949; 4,294,832; and 4,350,704.

Lisinopril, (S)-1-[N²-(1-(carboxy-3-phenylpropyl)-L-lysyl]-L-proline, is an ACE inhibitor described in U.S. Pat. No. 4,555,502. Following oral administration, peak serum concentrations of lisinopril occur within about 7 hours, although there was a trend to delay such peak serum concentrations in acute myocardial infarction patients. Lisinopril does not undergo metabolism and is excreted unchanged in the urine. The antihypertensive action of lisinopril is believed to result primarily from the suppression of the renin-angiotensin system as a result of inhibition of angiotensin II formation. The recommended starting dosage of lisinopril as monotherapy for essential hypertension is 10 mg once per day, with drug titration 20 to 40 mg per day. Dosages up to 80 mg per day have been used but do not appear to give a greater effect. The most common dosage is 20-40 mg per day. Several weeks of therapy may be required to achieve optimal blood pressure reduction for a patient. For patients with renal impairment, the recommended initial dose is 2.5 mg. The dosage may be titrated upward until blood pressure is controlled or to a maximum of 40 mg daily. Lisinopril is commercially available from pharmaceutical suppliers (e.g., AstraZeneca (sold under the trade name Zestril®) and Merck (sold under the trade name Prinvil®)) and has been approved for treatment of hypertension in several countries. Lisinopril has various side-effects including headache, dizziness, fatigue, cough, gastrointestinal disturbance, upper respiratory infection, diarrhea, muscle cramps, rash, and impotence.

Another class of active agents that is used for the treatment of hypertension is calcium antagonists. These active agents influence the influx of calcium ions into cells, especially smooth muscle cells. Inhibition of calcium influx produces a relaxation of smooth muscles, including those around the arteries and veins, which leads to a decrease in observed hypertension. Calcium antagonists and their hypotensive activity are described in a number of publications and patent applications.

Lercanidipine (methyl 1,1,N-trimethyl-N-(3,3-diphenylpropyl)-2-aminoethyl 1,4-dihydro-2,6-dimethyl-4-(3-nitrophenyl)pyridine-3,5-dicarboxylate) is a highly lipophilic dihydropyridine calcium antagonist with long duration of action and high vascular selectivity. The mechanism of lercanidipine's antihypertensive activity is due to a direct relaxant effect on vascular smooth muscle, thus lowering total peripheral resistance. The recommended starting dose of lercanidipine as monotherapy is 10 mg daily by oral route, with a drug titration to 20 mg daily (for immediate release). Daily administration of up to 80 mg per day is permitted for novel modified-release formulations of lercanidipine, which, due to the modified release profile, prevent excessive plasma levels of lercanidipine at any given time point. When administered as an unmodified release oral dosage form, lercanidipine is rapidly absorbed with peak plasma levels occurring 2-3 hours following dosing. Elimination is essentially via the hepatic route. By virtue of its high lipophilicity and high membrane partition coefficient, lercanidipine combines a short plasma half life with a long duration of action. The preferential distribution of lercanidipine into membranes of smooth muscle cells results in membrane-controlled pharmacokinetics that is characterized by a prolonged pharmacological effect. In comparison to other calcium antagonists, lercanidipine is characterized by gradual onset and long-lasting duration of action, despite decreasing plasma levels. In vitro studies show that isolated rat aorta response to high K⁺ may be attenuated by lercanidipine, even after the drug has been removed for 6 hours.

Lercanidipine has been found to have a renal-protective effect. Sabbatini, et al. Hypertension, 200, 35:775-779; Hayashi, et al., in Epstein, M. Calcium Anatagonists in Clinical Medicine, 3d ed., New York, Hanley & Belfus, 2001, pp. 559-578.

Lercanidipine is commercially available from Recordati S.p.A. (Milan, Italy) and has been described along with methods for making it and resolving it into individual enantiomers in U.S. Pat. Nos. 4,705,797; 5,767,136; 4,968,832; and 5,696,139.

Clinical studies have shown that lercanidipine 10 mg daily (typically titrated to 20 mg daily in patients not responding or responding inadequately to the 10 mg dose) provides a sustained pharmacological action and a significant antihypertensive effect. In hypertensive patients, the onset of lercanidipine action is gradual and the drug has a consistent and sustained blood pressure lowering effect throughout the dosage interval. The gradual and smooth antihypertensive effect has been confirmed by using the “Smoothness Index”, as described in Omboni and Zanchetti, Hypertension, 1998, 16:1831-8. The analysis of a large population of hypertensive patients has documented that lercanidipine is a very well tolerated drug devoid of major side effects, and has a lower frequency and intensity of side effects than other dihydropyridines as reported in Bang et al., Drugs, 2003, 63:2449-72. No alarming signals on safety or drug-interactions have emerged for lercanidipine, indicating that its use in hypertensive patients may be considered safe. In humans, lercanidipine is contraindicated in patients with unstable angina or recent (<1 month) myocardial infarction (as are all other dihydropyridines).

Several pharmacological rationales have been used for combining an ACE-inhibitor and a calcium antagonist to treat hypertension. For example, the fact that multiple physiologic systems participate in blood pressure control has been proposed as the reason why individual active agents decrease in efficacy over time. The pharmacological intervention in one of these systems is believed to trigger counterregulatory mechanisms. A combination of treatments increases the number of mechanisms potentially capable of reducing an elevated blood pressure and reduces the rate and magnitude of the adverse events produced by each drug. Further, the addition of one agent may counteract some deleterious effect of the other. Therefore a low-dose combination of two different agents reduces the risk of dose-related adverse reaction while still allowing sufficient blood pressure reduction. Optimal associations are those between a thiazide diuretic and an ACE-inhibitor or a calcium antagonist and an ACE-inhibitor. Associations between a calcium antagonist and a diuretic or between an ACE-inhibitor and a beta-blocker also can be used, but partial overlap of their mechanism of action may make their effectiveness less than the sum of individual agents (Mancia and Grassi, High Blood Pressure 1994; 3 (Suppl.4): 5-7).

The ACE-inhibitors attenuate vasoconstriction through reduction of the vasoconstrictive effect of angiotensin II and augmentation of the vasodilatatory kinins, whereas the calcium antagonists act through attenuating the transmembrane flux of calcium inhibit calcium-mediated electromechanical coupling in contractive tissue in response to numerous stimuli. Moreover, both classes of drugs facilitate salt and water excretion by the kidney through different mechanisms. ACE-inhibitors restore the renal-adrenal response to salt loading, whereas calcium antagonists possess intrinsic natriuretic properties, probably through a mechanism of inhibiting tubular salt and water reabsorption (Weir, AJH 1998; 11: 163S-169S).

ACE-inhibitors also may reduce the counterregulatory effects induced by calcium antagonists (i.e., stimulation of the sympathetic system). The negative sodium balance induced by calcium antagonists could potentiate the hypotensive effects of ACE-inhibitors (Menard and Bellet J. Cardiovasc. Pharmacol 1993; 21 (Suppl.2):S49-S54).

In addition to pharmacological advantages, combination therapy has been requested to meet evolving guidelines that look for more aggressive treatment of blood pressure. For example, recent World Health Guidelines recommend a diastolic blood pressure lower than 85 mm Hg and a systolic blood pressure lower than 130 mm Hg in younger patients and in diabetic patients.

Currently, there are various fixed combinations of ACE-inhibitors and calcium antagonists that are marketed in Europe and in the United States. These include combinations of ramipril and felodipine, trandolapril and verapamil, enalapril and felodipine, benazepril and amlodipine, and enalapril and diltiazem. Many patients may experience side effects due to one or both of the administered active agents, or due to the specific combination of the two active agents. However, fixed combinations offer the possibility of administering a combination of active agents in a single dosage form. Such a form will likely increase patient compliance. That is, such a dosage form will likely increase a patient's adherence to a therapeutic scheme and will increase the success of such the treatment therapy. Moreover, once the appropriate dosages of each ingredient are worked out, the appropriate combination formulation can be administered as a single dosage form, if desired.

Additionally, a number of patents are either nonresponsive to one or more of the available monotherapies, and some patients are not responsive to the aforementioned combination therapies. There is no way at present to predict whether these patients will be responsive to therapy using a new combination of active ingredients. It has been calculated that, overall, 30-50% of patients are non-responders to monotherapy (this average does not include data of patients taking lercanidipine).

Single dosage forms of lisinopril and the diuretic hydrochlorothiazide are approved for treatment of hypertension and are commercially available from AstraZeneca (Zestoretic®) and Merck (Prinzide®). The available dosage forms comprise 10 mg lisinopril and 12.5 mg hydrochlorothiazide, 20 mg lisinopril and 12.5 mg hydrochlorothiazide, 20 mg lisinopril and 25 mg hydrochlorothiazide. Accordingly, the available dosage form included quantities of lisinopril that are typically sufficient in once a day monotherapy.

Therefore, there is a continuing need for effective combination anti-hypertensive treatments that have a long lasting, selective mechanism of action with few side effects.

### BRIEF DESCRIPTION OF THE DRAWINGS

FIG. 1 depicts the effect of renal ischemia on diastolic blood pressure (DBP) in uninephrectomized anesthetized rats treated with vehicle, lercanidipine (10 μg/kg), lisinopril (30 μg/kg), and the combination of lercanidipine and lisinopril.

FIG. 2 depicts the effect of renal ischemia on systolic blood pressure (SBP) in uninephrectomized anesthetized rats treated with vehicle, lercanidipine (10 μg/kg), lisinopril (30 μg/kg), and the combination of lercanidipine and lisinopril in the same rat model and experiment of FIG. 1.

FIG. 3 depicts the effect of drugs administration (lercanidipine individually, lisinopril/HCTZ in dual combination, and all three in triple combination) on systolic blood pressure plotted against time in renal ischemic uninephrectomized anaesthetized rats.

FIG. 4 depicts effects of administration of same drugs (individually for lercandipine, in dual combination for lisinopril/HCTZ or all three in triple combination) as in FIG. 3 on diastolic blood pressure plotted against time in the same rat model and experiment of FIG. 3.

FIG. 5 depicts the change in systolic blood pressure following administration of same drugs as in FIG. 3 (as the difference between SBP at given times and the SBP at the time of administration) tested in the same animal model (administration time: t=0).

FIG. 6 depicts the decrease in diastolic blood pressure after administration of the same drugs as in FIG. 3 to the same animal model plotted against the difference between DBP at given times and the DBP at the time of administration (administration time: t=0).

FIG. 7 depicts the effect of administration of the same drugs as in FIG. 3 plotted as a function of time on heart rate in the same animal model.

FIG. 8 depicts the effect on heart rate change of administration of the same drugs as in FIG. 3 plotted as the difference between heart rate at given times and the heart rate at the time of administration, tested in the same animal model (administration time: t=0).

### SUMMARY OF THE INVENTION

The present invention contemplates methods for treating hypertension by administering a combination comprising lisinopril and lercanidipine in four classes of patients. The first class of patients are those that are responders to monotherapy with either lisinopril (or dual therapy of lisinopril plus HCTZ) or lercanidipine, but who suffer from side-effects and for whom it would be desirable to decrease the dosage amount of the active agent used in monotherapy (or in dual lisinopril HCTZ therapy). In other words, these active agents produce antihypertensive activity and decrease the patient's blood pressure by the predetermined increment. A combination comprising of lisinopril and lercanidipine is particularly suitable for such patients.

Accordingly, in one aspect, the present invention is directed to a method for treating hypertension in a patient in need thereof, the method comprising administering to the patient a first amount of lercanidipine and a second amount of lisinopril, where the amounts in combination are effective to reduce blood pressure in the patient by at least a predetermined increment and thus restore blood pressure to within acceptable limits; where at least one of the first amount and the second amount is either ineffective to produce a reduction in blood pressure in the patient, or the reduction in blood pressure is less than the predetermined increment. In other words, the amounts of the two agents employed in the combination would each be suboptimal or sub-threshold (i.e., producing a decrease in blood pressure less than the predetermined amount or totally ineffective if administered as monotherapy or as lisinopril/HCTZ dual therapy). In a preferred embodiment both the first amount and the second amount are ineffective to produce a reduction in blood pressure in the patient, or the reduction in blood pressure is less then the predetermined amount.

The second patient class are of patients who are “nonresponders” to monotherapy (or to dual therapy using HCTZ and lisinopril). In these patients, the active agent or agents alone do not produce antihypertensive activity. In another aspect, the present invention encompasses a method for treating hypertension in a nonresponder patient in need thereof, the method comprising administering to the patient a first amount of lercanidipine and a second amount of lisinopril, where the amounts in combination are-effective to reduce blood pressure in the patient by at least a predetermined increment, and thus restore blood pressure to within acceptable limits. The patient would usually have been previously determined not to respond or to respond insufficiently to monotherapy with lercanidipine or lisinopril, or even with another single antihypertensive agent. This embodiment is particularly desirable for those patients that are resistant to lercanidipine monotherapy. Lercanidipine generally works quite well, so patients resistant to lercanidipine monotherapy can be difficult to treat.

The third class of patients are of patients who are partial responders to monotherapy and/or combination therapy (using lisinopril/HCTZ or other dual combinations involving or not involving lercanidipine). Monotherapy or combination therapy produces an antihypertensive effect in these patients, but the therapy does not decrease the blood pressure by the predetermined increment. Higher doses do not produce the desired effect of decreasing blood pressure by the predetermined amount, and may produce undesirable side effects. In another aspect, the present invention encompasses a method for treating hypertension in a partial responder patient in need thereof, the method comprising administering to the patient a first amount of lercanidipine and a second amount of lisinopril, wherein the amounts in combination are effective to reduce blood pressure in the patient by at least a predetermined increment, wherein each of the first amount and the second amount if administered alone is ineffective to produce a reduction in blood pressure by the predetermined increment.

The fourth class of patients includes those that are responders to monotherapy (or to dual therapy of lisinopril and HCTZ) but have been previously determined (or are expected) to become nonresponders over time. Conventionally, patients in this class, upon becoming nonresponders, would then require a monotherapy involving higher dosage amounts of the same active agent or would need a change of medication to another active agent to treat hypertension (i.e., reduce blood pressure by the predetermined increment). However, it should be noted that these patients may not further respond to increased dosages due to maximal efficacy of the compound having been reached. The cause for such a change in a patient's response also may be a compensation (counterregulatory) mechanism or another cause.

In yet another aspect, the present invention encompasses a method for treating hypertension in a patient within the fourth class, where the patient has been previously determined to be responsive to monotherapy with lercanidipine or with lisinopril (or with another monotherapy or with lisinopril/HCTZ double therapy), the method comprising administering to the patient a composition comprising a first “dual combination therapy amount” of lercanidipine and a second double combination therapy amount of lisinopril, where the dual combination therapy amounts are in combination effective to reduce the patient's blood pressure by at least the predetermined increment, and thus restore blood pressure to within acceptable limits. In a preferred embodiment, the amounts of lisinopril and lercanidipine are sub-threshold amounts of each agent that would not be effective in monotherapy.

Lastly, in principle, the present invention can be employed with naive patients although the regulatory authorities guidelines do not encourage such a practice.

In a separate embodiment the present invention provides a method for treating hypertension in a patient in need of treatment (whether in one of the foregoing classes of patents or not) by administering to the patient antihypertensive triple combination (in single dosage forms or in two dosage forms or in three dosage forms) but preferably in a single dosage form) comprising a first triple combination amount of lercanidipine a second triple combination amount of lisinopril and a third triple combination amount of a diuretic such as HCTZ, the amount in combination being effective in reducing blood pressure in a patient in need of such treatment, preferably reducing said blood pressure (the diastolic or the systolic component thereof or both) by a predetermined increment (or, for both components, by first and second predetermined increments). Preferably, at least one of the first, second or their triple combination amounts in this triple combination is suboptimal or subthreshold. In another aspect of preference, at least two of said amounts are suboptimal.

In yet another aspect, the present invention encompasses methods of treating a patient within the aforementioned classes of patients with a double or with a triple combination of the invention wherein said patient is a diabetic (e.g., a type II diabetic), even when the diabetic patient has proteinuria, or wherein the patient has otherwise compromised renal function. In particular, it is anticipated that the triple combination of the present invention will permit even some patients with compromised renal function such as diabetes, to achieve better control of their blood pressure through the administration of a diuretic (such as HCTZ) in addition to the lisinopril/lercanidipine combination, without additionally compromising their renal function or presenting with excessive proteinura.

Compositions and dosage forms are further contemplated by the present invention to implement the foregoing methods.

The compositions and methods described herein have the potential advantages of allowing treatment with sub-threshold amounts of at least one active agent, allowing greater tolerability in patients sensitive to the active agent, of allowing for synergism, i.e., superadditivity between active agents (demonstrated herein for the triple combination), of allowing for sustained long term efficacy of treatment and for sustained dosing throughout a dosage period.

### DETAILED DESCRIPTION OF PREFERRED EMBODIMENTS

As used herein, the term “hypertension” refers to abnormally high arterial blood pressure, when compared to prior blood pressure readings, and the abnormally high value is maintained over a specified time period. Conventionally, the time period is 3-6 months. The increase may be observed in systolic pressure, diastolic pressure, or both. Conventionally, hypertension is defined as a blood pressure of equal to or greater than 140/90 mm Hg. Blood pressure may be measured by any method known in the art. Such methods include, but are not limited to direct arterial puncture, oscillometry, Doppler ultrasonography, and a sphygmomanometer. In a preferred embodiment, blood pressure is measured with a sphygmomanometer. While the person taking the measurement listens to the pulse of the patient and watches the sphygmomanometer gauge, two measurements (systolic pressure and diastolic pressure) are recorded. Blood pressure is measured in millimeters of mercury (mm Hg). A more refined version of the nomenclature for hypertension is provided at the table below:

Blood pressure in normal and hypertensive adults is typically categorized as follows:

| | Systolic | Diastolic |
|---|---|---|
| | Pressure, | Pressure, |
| Category | mmHg | mmHg |
| Normal | <120 | <80 |
| Prehypertension | 120-139 | 80-89 |
| Stage 1 Hypertension | 140-159 | 90-99 |
| Stage 2 Hypertension | >160 | >100 |

Source:
The Seventh Report of the Joint National Committee on Prevention, Detection, Evaluation, and treatment of High Blood Pressure (JAMA, 289 (19) 2560-72 (2003))

The terms “systolic” and “systolic pressure” refer to the pressure induced by the contraction of the heart by which the blood is forced onward and the circulation kept up. The terms “diastolic” and “diastolic pressure” refer to the pressure observed during the dilatation of the cavities of the heart, during which they fill with blood. Typically, blood pressure is expressed as two numbers separated by a slash, where the first number is the systolic pressure and the second number is the diastolic pressure. As mentioned above, the pressure is conventionally expressed as mm Hg.

The term “antihypertensive activity” refers to the effect of an active agent to lower the blood pressure of a patient with hypertension. In one embodiment, the blood pressure is decreased by at least 20 mm Hg for systolic pressure or by at least 10 mm Hg for diastolic pressure. In another embodiment, the antihypertensive activity refers to the effect of an active agent to lower the blood pressure by at least 20 mm Hg for systolic pressure and by at least 10 mm Hg for diastolic pressure. The active agent may or may not decrease the blood pressure in a person that does not have hypertension or may not decrease blood pressure in all persons with hypertension. In a preferred embodiment, the active agent decreases a patient's blood pressure to below 140/90 mm Hg.

The term “active agent” or “active ingredient” refers to a compound that produces a pharmacological effect that leads to a physiological change. As used herein, the active agents are antihypertensive agents, such as lercanidipine and lisinopril, which are employed in the combination treatment of the invention. Conventionally, an active agent is considered as having an antihypertensive effect if it decreases either systolic or diastolic blood pressure by at least 10 mm Hg.

The term “predetermined increment” refers to the minimum reduction in blood pressure that is needed for a patient to decrease blood pressure to or below 140/90. Thus, an active agent which at a dosage tolerated by the patient achieves reduction by a predetermined increment is considered effective to treat hypertension in the specific patient, and the patient is considered responsive to this agent (also known as a “responder”). In other words, if an active agent decreases blood pressure by a predetermined increment in one patient (i.e., has sufficient antihypertensive activity in the patient) but does not decrease blood pressure by the predetermined increment in another patient (i.e., does not have sufficient antihypertensive activity in the patient), then the first patient is responsive to the treatment (a “responder”, as defined below) but the second patient is not (a “nonresponder”, as defined below). The decrease in blood pressure can be in the systolic pressure, diastolic pressure, or both.

As used herein, the term “responder” refers to a patient that has previously responded to a treatment for hypertension involving administration of a particular active agent (or combination of active agents) in a particular amount or amounts. In other words, the active agent or active agents have “antihypertensive activity” and reduce the patient's blood pressure by the “predetermined increment”. A determination of responsiveness to an antihypertensive regimen may require administration of a particular agent in a particular amount and frequency for a period of time, usually 1 month for ACE inhibitors and calcium antagonists. Such treatments include, but are not limited to, administration of ACE inhibitors, calcium channel blockers, beta blockers, and diuretics. The phrase “responsive to monotherapy” refers to patients who are administered only one active agent (monotherapy) and the monotherapy achieves a reduction in blood pressure by the “predetermined increment” as that term is defined above. In a specific embodiment, the antihypertensive activity is defined as at least a decrease of 20 mm Hg in systolic pressure or as at least a decrease of 10 mm Hg for diastolic pressure.

The term “nonresponder” refers to a patient who has been determined not to have responded to treatment for hypertension with a particular agent or combination of agents, i.e., for whom the regimen has not achieved a reduction in blood pressure. In other words, the active agent or active agents do not have “antihypertensive activity” in the patient, and therefore the patient's blood pressure is not decreased by the “predetermined increment”. The term encompasses patients that do not undergo any decrease in blood pressure upon treatment e.g. with lercanidipine alone or lisinopril alone.

The term “partial responder” refers to a patient for whom a particular active agent (or combination of active agents), in a particular amount or amounts, produces “antihypertensive activity” in the patient but does not decrease blood pressure by the “predetermined increment”. Increases in the amount of active agent (or combination of active agents) may or may not further decrease the blood pressure of these patients. The term encompasses patients that respond only insufficiently, i.e., exhibit some decrease in blood pressure, but short of the “predetermined increment” (to below 140/90 mm Hg). Generally, in those patients the amount of antihypertensive agent needs to be increased. But this may bring on or aggravate side effects.

The terms “suboptimal” or “sub-threshold” amounts of active agent for monotherapy refer to amounts of active agent that insufficient to decrease blood pressure by the predetermined increment. “Suboptimal” or “sub-threshold” amounts may well vary from patient to patient. A patient who fails to achieve a decrease in blood pressure by the predetermined increment upon administration of a given dosage of active agent has either been administered a “suboptimal” or “sub-threshold” amount of active agent or may, alternatively, be a non-responder to the active agent. “Suboptimal” or “sub-threshold” amounts of active agent may be distinguished from the case of administration to a non-responder by increasing the administered dosage of active agent. In the case where a patient fails to achieve a decrease in blood pressure by the predetermined increment due to administration of a “suboptimal” or “sub-threshold” amount of active agent, administration of an increased dosage of active agent will cause the patient to achieve a decrease in blood pressure by the predetermined increment. In the case where a patient fails to achieve a decrease in blood pressure by the predetermined increment due to said patient being a non-responder, increasing the dosage of active agent will not cause the patient to achieve a decrease in blood pressure by the predetermined increment.

As used herein, the term “monotherapy” refers to the administration of a single active agent to treat hypertension.

The term “efficacy of treatment” refers to the potency of a drug in treating hypertension.

The term “in combination” refers to the concomitant administration of two (or more) active agents for the treatment of a single disease state. As used herein, the active agents may be combined and administered in a single dosage form, may be administered as separate dosage forms at the same time, or may be administered as separate dosage forms that are administered alternately or sequentially on the same or separate days. In one preferred embodiment of the present invention, the active agents are combined and administered in a single dosage form. In another preferred embodiment, the active agents are administered in separate dosage forms (e.g., wherein it is desirable to vary the amount of one but not the other). The single dosage form may include additional active agents for the treatment of the disease state. In a preferred embodiment, the single dosage form comprises lercanidipine and lisinopril.

The term “combination therapy” refers to administration of at least two active ingredients “in combination” i.e., as part of the same therapeutic regimen or dosage form for the treatment of hypertension. In the present invention lercanidipine and lisinopril may be further combined with one or more additional active ingredients, e.g., a diuretic and/or a β-receptor blocker and/or an angiotensin II receptor antagonist, without limitation. In the latter context, the terms “double combination” and “triple combination” when used in connection with embodiments of the present invention refer to combinations of (i) lercanidipine and lisinopril or (ii) lercinidipine/lisinopril/diurectic, respectively.

In the present invention, the amount of lisinopril administered to a patient in the double or triple combination therapy will be preferably within the range of 2.5 to 40 mg per day in a single or two divided doses. Lisinopril is marketed in dosages of 2.5 and 5 mg as starting dosages for patients on dialysis or who have other renal problems. Such patients are started on these low dosages of lisinopril prior to increasing dosages to effective amounts. Hence, 2.5 and 5 mg dosages of lisinopril are typically suboptimal or sub-threshold amounts that are not effective for control of hypertension. More preferably, the amount of lisinopril will be 10-20 mg per day. The amount of lercanidipine will be preferably within the range of 2.5-80 mg, more preferably, 10-20 mg.

The most preferred double combinations are: (i) 2.5 mg of lisinopril and 2.5 mg of lercanidipine, (ii) 2.5 mg of lisinopril and 5 mg of lercanidipine, (iii) 2.5 mg lisinopril and 10 mg lercanidipine, (iv) 2.5 mg lisinopril and 20 mg lercanidipine, (v) 2.5 mg of lisinopril and 40 mg lercanidipine, (vi) 2.5 mg of lisinopril and 60 mg lercanidipine, (vii) 2.5 mg of lisinopril and 80 mg lercanidipine, (viii) 5 mg of lisinopril and 2.5 mg of lercanidipine, (ix) 5 mg of lisinopril and 5 mg of lercanidipine, (x) 5 mg of lisinopril and 10 mg of lercanidipine, (xi) 5 mg lisinopril and 20 mg lercanidipine, (xii) 5 mg of lisinopril and 40 mg of lercanidipine, (xiii) 5 mg of lisinopril and 60 mg of lercanidipine, (xiv) 5 mg of lisinopril and 80 mg of lercanidipine, (xv) 10 mg lisinopril and 2.5 mg lercanidipine, (xvi) 10 mg of lisinopril and 5 mg of lercanidipine, (xvii) 10 mg of lisinopril and 10 mg of lercanidipine, (xviii) 10 mg lisinopril and 20 mg lercanidipine, (xix) 10 mg lisinopril and 40 mg lercanidipine, (xx) 10 mg lisinopril and 60 mg lercanidipine, (xxi) 10 mg lisinopril and 80 mg lercanidipine, (xxii) 20 mg lisinopril and 2.5 mg lercanidipine, (xxiii) 40 mg lisinopril and 5 mg lercanidipine, (xxiv) 20 mg lisinopril and 10 mg lercanidipine, (xxv) 20 mg of lisinopril and 20 mg of lercanidipine, (xxvi) 20 mg of lisinopril and 40 mg of lercanidipine, (xxvii) 20 mg of lisinopril and 60 mg of lercanidipine, (xviii) 20 mg of lisinopril and 80 mg of lercanidipine, (xxix) 40 mg lisinopril and 2.5 mg lercanidipine, (xxx) 40 mg lisinopril and 5 mg lercanidipine, (xxxi) 40 mg lisinopril and 10 mg lercanidipine, (xxxii) 40 mg of lisinopril and 20 mg of lercanidipine, (xxxiii) 40 mg of lisinopril and 40 mg of lercanidipine, (xxxiv) 40 mg of lisinopril and 60 mg of lercanidipine, or (xxxv) 40 mg of lisinopril and 80 mg of lercanidipine. Amounts may need to be optimized according to the needs of particular patient subpopulations depending on whether they are responders, partial responders, nonresponders, or naive to lercanidipine and/or lisinopril monotherapy at a tolerated dose. (In the case of naive patients, the starting amounts of the combination may be even smaller than the indicated dose, e.g., 2.5 mg of lisinopril).

Diuretics can be thiazide diuretics, potassium sparing diuretics or loop diuretics.

Thiazide diuretics directly inhibit sodium and calcium reabsorbtion and augment calcium absorption in the early distal convoluted tubule of the kidney. Excess sodium, calcium, and water reduce extracellular volume in mild to moderate congestive heart failure. Thiazide diuretics potentiate anti-hypertensive agents by about ⅓ to ½ in an effort to reduce blood pressure and overall fluid volume. The increased serum concentrations of calcium allow for the relaxation of arterial smooth muscle that, in turn, reduces peripheral vascular resistance. Thiazide diuretics possess anti-hypertensive properties because of the direct vasodilatation of arterioles, altered sodium balance, and the reduction in fluid volume. Thiazide diuretics are not the drug of choice if massive amount of diuresis are necessary or if the patient has a history of intolerance to sulfa containing medications.

Commonly used thiazide diuretics in the United States are Aquatensen® (methyclothiazide), Diucardin® (hydroflumethiazide), Diulo® (metolazone), Diuril® (chlorothiazide), Enduron® (methyclothiazide), Esidrix® (hydrochlorothiazide), Hydro-chlor® (hydrochlorothiazide; HCT), Hydro-D® (hydrochlorothiazide; HCT), HydroDIURIL® (hydrochlorothiazide), Hydromox® (quinethazone), Hygroton® (chlorthalidone), Metahydrin® (trichlormethiazide), Microzide® (hydrochlorothiazide), Mykrox® (metolazone), Naqua® (trichlormethiazide), Naturetin® (bendroflumethiazide), Oretic® (hydrochlorothiazide), Renese® (polythiazide), Saluron® (hydroflumethiazide), Thalitone® (chlorthalidone), Trichlorex® (trichlormethiazide), and Zaroxolyn® (metolazone). An additional thiazide or thiazide-like diuretic includes Lozol® (indapamide), benzylhydrochlorothiazide, cyclopenthiazide, polythiazide, ethiazide, and cyclothiazide.

Preferred diuretics are, without limitation, hydrochlorothiazide (HCT), 6-chloro-3,4-dihydro-2H-1,2,4,-benzothiadiazin-7-sulfonamide 1,1-dioxide and chorthalidone.

Potassium-sparing diuretics are mild diuretics that act upon the distal convoluting tubule to inhibit sodium exchange for potassium. Gradually, sodium and water are excreted in the urine, and potassium is conserved. Aldactone (spironolactone) is a synthetic steroid that is similar to aldosterone and acts as an antagonist by competing for aldosterone binding sites. Inhibition of aldosterone leads to the excretion of sodium and the retention of potassium in the distal portion of the kidney nephrons. Other members of the potassium-sparing diuretic group do not alter aldosterone binding, but work primarily by impairing the exchange of potassium and sodium in the distal convoluting tubule. These agents act as a slight anti-hypertensive and potentiate anti-hypertensive medications. Potassium-sparing diuretics can be given in conjunction with potassium-pitching diuretics in an attempt to prevent hypokalemia and the complications involved with that particular electrolyte imbalance.

Commonly used potassium-sparing diuretics used in the United States include Aldactone® (spironolactone), Dyrenium® (triamterene), and Midamor® (amiloride). More recently, a more selective antagonist of the mineralocorticoid receptor, Inspra® (eplerenone) has been introduced.

Loop diuretics relieve excess extracellular fluid volume and to regulate vascular osmolarity. Loop diuretics are the most potent and expedient diuretics available, and they inhibit the reabsorbtion of sodium, chloride, and potassium ions in the ascending loop of Henle in the kidney. Loop diuretics also cause renal vasodilatation and a transient rise in glomerular filtration rate. The combination of increased renal blood flow and the prevention of the sodium-potassium-chloride co-transport system permits secretion of large volumes of fluid and electrolytes. Loop diuretics have systemic hemodynamic effects: increased venous capacitance (which reduces left ventricular filling pressures or preload), increased ejection fraction (an indicator of improved ventricular function), decreased systemic and peripheral vascular resistance (reduced pulmonary, organ, and extracellular edema or afterload) which all allow for a reduction in blood pressure and cardiac workload.

Commonly used brand names in the United States are bumetamide (Bumex®, Budema®, Bumedyl®, Burinex®, Busix®, Butinat®, Cambiex®, Farmadiuril®, Fontego®, Fordiuran®, Lunetoron®, Miccil®, Pendock®, Poliurene®, Segurex®); torsemide (Demadex®); ethacrynic acid (Edecrin®, Edecril®, Hydromedin®, Reomax®); furosemide (Lasix®, Myrosemide®, Aldic®, Aluzin®, Aquamide®, Aquasin®, Arasemide®, Bioretic®, Cetasix®, Detue®, Dirine®, Discoid®, Disemide®, Diural®, Diuresal®, Diurolasa®, Diusil®, Dranex®, Dryptal®, Durafurid®, Edenol®, Errolon®, Eutensin®, Fluidrol®, Franyl®b, Frumex®, Frusedan®, Frusema®, Frusemid®, Frusetic®, Frusid®, Frusix®, Furantril®, Furetic®, Furex®, Furmide®, Furocot®, Furodiurol®, Furomide M.D®, Furorese®, Furoside®, Furosix®, Furovite®, Fusid®, Golan®, Hissuflux®, Koftizo®, Kutrix®, Lasemid®, Lasiletten®, Lasilix®, Laxur®, Liside®, Lo-Aqua®, Luramide®, Marsemid®e, Nadis®, Nelsix®, Nildema®, Novosemide®, Odemase®, Oedemex®, Promedes®, Radisemide®, Radonna®, Rasitol®, Retep®, Ro-Semide®, Rose-40®, Salinex®, Salurid®, Seguril®, Sigasalur®, Trofurit®, Uremide®, Urenil®, Uresix®, Urian®, Uritol®, Yidoli, Furosan®, and Furoter®).

The amounts of diuretic vary according to the diuretic employed. For HCTZ the usual preferred amount is 12.5 or 25 mg/per day (in one or in divided doses) when the HCTZ is administered together with another active ingredient such as lisinopril. In principle, however, the amount of HCTZ can be as low as 8 mg/day or ever lower, e.g. 5 mg/day, and as high as 100 mg/day. (In certain patient subpopulations, the amount of HCTZ may be kept low, below 25 or even below 12 mg, e.g., in patients with compromised renal function.) For immediate release triple combinations, a rule of thumb for determining HCTZ amount is 1.2 parts HCTZ per part lisinopril (by weight). For sustained release formulations and more generally all for formulation containing higher amounts of lercanidipine, adjustments may be necessary as known by and appreciated by those skilled in the art.

The most preferred triple combinations simply add HCTZ as follows:
3.0 mg to double combination (i) above;
6.0 mg to (ii); 12 mg to (iii); 25 mg to (iv); from 25 mg to about 50 mg to (v); from 25 mg to about 70 mg to (vi); from 25 mg to about 100 mg to (vii). Triple combinations based on double combinations (viii) through (xiv), (xv) through (xxi), (xxii) through (xviii) and (xxix) through (xxxv) will contain the same amounts of HCTZ written for triple combinations based on double combinations (i) through (vii) which contain the same amount of lercanidipine. Thus, a triple combination based on double combinations (xi) will contain 25 mg of HCTZ just like the triple combination based on double combination (iv).

It has been found that triple combination treatment with lercanidipine, lisinopril, and a diuretic, e.g., HCTZ, leads to an unexpectedly augmented superior treatment of hypertension, i.e., an unexpectedly superior reduction in blood pressure, compared to monotherapies or double combination therapies.

### Pharmaceutical Compositions

The active agents of the combination of the present invention may be formulated into a single pharmaceutical composition or each can be administered in a different pharmaceutical composition, or in the case of triple combinations in one, two or three compositions. In any case, the pharmaceutical composition also may include optional additives, such as a pharmaceutically acceptable carrier or diluent, a flavorant, a sweetener, a preservative, a dye, a binder, a suspending agent, a dispersing agent, a colorant, a disintegrant, an excipient, a diluent, a lubricant, a plasticizer, an edible oil or any combination of two or more of the foregoing.

Suitable pharmaceutically acceptable carriers or diluents include, but are not limited to, ethanol; water; glycerol; aloe vera gel; allantoin; glycerin; vitamin A and E oils; mineral oil; PPG2 myristyl propionate; magnesium carbonate; potassium phosphate; vegetable oil; animal oil; and solketal.

Suitable binders include, but are not limited to, starch; gelatin; natural sugars, such as glucose, sucrose and lactose; corn sweeteners; natural and synthetic gums, such as acacia, tragacanth, vegetable gum, and sodium alginate; carboxymethylcellulose; polyethylene glycol; waxes; and the like.

Suitable disintegrators include, but are not limited to, starch such as corn starch, methyl cellulose, agar, bentonite, xanthan gum and the like.

Suitable lubricants include, but are not limited to, sodium oleate, sodium stearate, magnesium stearate, sodium benzoate, sodium acetate, sodium chloride and the like.

A suitable suspending agent is, but is not limited to, bentonite.

Suitable dispersing and suspending agents include, but are not limited to, synthetic and natural gums, such as vegetable gum, tragacanth, acacia, alginate, dextran, sodium carboxymethylcellulose, methylcellulose, polyvinyl-pyrrolidone and gelatin.

Suitable edible oils include, but are not limited to, cottonseed oil, sesame oil, coconut oil and peanut oil.

Examples of additional additives include, but are not limited to, sorbitol; talc; stearic acid; and dicalcium phosphate. Commercially available preparations containing lisinopril (with or without another active ingredient) and lercanidipine can be used. Naturally, if the preparation contains more than one active ingredient, the amounts in the combination may have to be adjusted downwards.

The amount of active agent(s) can vary from about 0.01% to about 100% of the dosage form, based upon the weight of the dosage form.

Unit Dosage Forms

The pharmaceutical composition may be formulated as unit dosage forms, such as tablets, pills, capsules, boluses, powders, granules, sterile parenteral solutions, sterile parenteral suspensions, sterile parenteral emulsions, elixirs, tinctures, metered aerosol or liquid sprays, drops, ampoules, autoinjector devices or suppositories. Unit dosage forms may be used for oral, parenteral, intranasal, sublingual or rectal administration, or for administration by inhalation or insufflation, transdermal patches, and a lyophilized composition. In general, any delivery of active ingredients that results in systemic availability of them can be used. Preferably the unit dosage form is an oral dosage form, most preferably a solid oral dosage, therefore the preferred dosage forms are tablets, pills, and capsules. However, parenteral preparations also are preferred.

Solid unit dosage forms may be prepared by mixing the active agents of the present invention with a pharmaceutically acceptable carrier and any other desired additives as described above. The mixture is typically mixed until a homogeneous mixture of the active agents of the present invention and the carrier and any other desired additives is formed, i.e., until the active agents are dispersed evenly throughout the composition. In this case, the compositions can be formed as dry or moist granules.

Tablets or pills can be coated or otherwise compounded to form a unit dosage form which has delayed and/or prolonged action, such as time release and sustained release unit dosage forms. For example, the tablet or pill can comprise an inner dosage and an outer dosage component, the latter being in the form of a layer or envelope over the former. The two components can be separated by an enteric layer which serves to resist disintegration in the stomach and permits the inner component to pass intact into the duodenum or to be delayed in release.

Biodegradable polymers for controlling the release of the active agents, include, but are not limited to, polylactic acid, polyepsilon caprolactone, polyhydroxy butyric acid, polyorthoesters, polyacetals, polydihydro-pyrans, polycyanoacrylates and cross-linked or amphipathic block copolymers of hydrogels.

For liquid dosage forms, the active substances or their physiologically acceptable salts are brought into solution, suspension or emulsion, optionally with the usually employed substances such as solubilizers, emulsifiers or other auxiliaries. Solvents for the active combinations and the corresponding physiologically acceptable salts, can include water, physiological salt solutions or alcohols, e.g. ethanol, propane-diol or glycerol. Additionally, sugar solutions such as glucose or mannitol solutions may be used. A mixture of the various solvents mentioned may further be used in the present invention.

A transdermal dosage form also is contemplated by the present invention. Transdermal forms may be a diffusion-driven transdermal system (transdermal patch) using either a fluid reservoir or a drug-in-adhesive matrix system. Other transdermal dosage forms include, but are not limited to, topical gels, lotions, ointments, transmucosal systems and devices, and iontohoretic (electrical diffusion) delivery system. Transdermal dosage forms may be used for timed release and sustained release of the active agents of the present invention.

Pharmaceutical compositions and unit dosage forms of the present invention for administration parenterally, and in particular by injection, typically include a pharmaceutically acceptable carrier, as described above. A preferred liquid carrier is vegetable oil. Injection may be, for example, intravenous, epidural, intrathecal, intramuscular, intraruminal, intratracheal, or subcutaneous.

The active agents also can be administered in the form of liposome delivery systems, such as small unilamellar vesicles, large unilamellar vesicles and multilamellar vesicles. Liposomes can be formed from a variety of phospholipids, such as cholesterol, stearylamine or phosphatidylcholines.

The active agents of the present invention may also be coupled with soluble polymers as targetable drug carriers. Such polymers include, but are not limited to, polyvinyl-pyrrolidone, pyran copolymer, polyhydroxypropylmethacryl-amidephenol, polyhydroxy-ethylaspartamidephenol, and polyethyl-eneoxideopolylysine substituted with palmitoyl residues.

Lercanidipine can be formulated as a physiologically acceptable salt, e.g., a salt with an inorganic or organic acid such as e.g. HCl, HBr, H₂SO₄, maleic acid, fumaric acid, tartaric acid and citric acid.

TABLE 1
Formulation I

| Ingredient | Amount (mg/tablet) |
|---|---|
| Lercanidipine HCl | 10 |
| Lisinopril (as dihydrate form) | 10 |
| Lactose | 102 |
| Microcrystalline cellulose | 40 |
| Sodium bicarbonate | 8 |
| Sodium starch glycolate | 20 |
| Povidone K30 | 8 |
| Magnesium stearate | 2 |

A film coated tablet may be prepared using the cores described above and using the composition described in Table 2.

TABLE 2
Coating for tablet formulation shown in Table 1.

| Ingredient | Amount |
|---|---|
| Hypromellose | 1.91 mg |
| Talc | 0.15 mg |
| Titanium dioxide | 0.60 mg |
| Macrogol 6000 | 0.30 mg |
| Ferric oxide | 0.04 mg |

TABLE 3
Formulation II

| Ingredient | mg/tablet | mg/tablet |
|---|---|---|
| Lercanidipine HCl | 5 mg | 10 mg |
| Lactose monohydrate | 35.0 mg | 30.0 mg |
| Microcrystalline cellulose | 39.0 mg | 39.0 mg |
| Sodium starch glycolate | 15.5 mg | 15.5 mg |
| Povidone | 4.5 mg | 4.5 mg |
| Magnesium stearate | 1.0 mg | 1.0 mg |
| Coating | | |
| Opadry OY-SR-6497 | | |
| Hypromellose | 1.91 mg | 1.91 mg |
| Talc | 0.15 mg | 0.15 mg |
| Titanium dioxide | 0.60 mg | 0.60 mg |
| Macrogol 6000 | 0.30 mg | 0.30 mg |
| Ferric oxide | 0.04 mg | 0.04 mg |
| Total | 103 mg | 103 mg |

If the foregoing also contain HCTZ, formulations contain 10 mg of lisinopril will contain 12 mg HCTZ and formulations including 5 mg of lisinopril will contain 6.0 mg HCTZ.

### Administration

The pharmaceutical composition or unit dosage forms of the present invention may be administered by a variety of routes such as intravenous, intratracheal, subcutaneous, oral, parenteral, buccal, sublingual, opthalmic, pulmonary, transmucosal, transdermal, and intramuscular. Unit dosage forms also can be administered in intranasal form via topical use of suitable intranasal vehicles, or via transdermal routes, using those forms of transdermal skin patches known to those of ordinary skill in the art. Oral administration is preferred.

The pharmaceutical composition or unit dosage forms of the present invention may be administered to an animal, preferably a human being, in need of antihypertensive treatment. The pharmaceutical composition or unit dosage form of the present invention may be administered according to a dosage and administration regimen defined by routine testing in light of the guidelines given above in order to obtain optimal antihypertensive activity (especially for patients who are partial responders or nonresponders to conventional monotherapy or to other combination therapies) and a decreased in blood pressure by the predetermined increment while minimizing toxicity or side-effects for a particular patient. However, such fine tuning of the therapeutic regimen is routine in light of the guidelines given herein.

The dosage of the active agents of the present invention may vary according to a variety of factors such as underlying disease state, the individual's condition, weight, sex and age and the mode of administration. For oral administration, the pharmaceutical compositions can be provided in the form of scored or unscored solid unit dosage forms. For lisinopril, the dosage forms comprise 2.5, 5, 10, 20, or 40 mg for the symptomatic adjustment of the dosage to the patient to be treated. Preferably, the lisinopril dosage forms comprise 2.5, 5, 10, or 20 mg. For lercanidipine, the dosage forms comprise 2.5, 5, 10, 20, 40, 60, or 80 mg for the symptomatic adjustment of the dosage to the patient to be treated. The 60 or 80 mg dosage is preferably administered only in the form of a modified release tablet. The 40 mg lercanidipine dosage form can be administered in either a modified release or in an unmodified release dosage form. The modified release decreases initial peak concentration and provides sustained blood levels for a prolonged period of time, e.g., 24 h. Preferably, the lercanidipine dosage forms comprise 2.5, 5, 10 or 20 mg. especially in immediate release formulations. The amount for HCTZ have been provided earlier in the present specification.

For both the double and triple combination therapy according to the invention, the active agents may initially be provided as separate dosage forms until an optimum dosage combination and administration regimen is achieved. Therefore, the patient may be titrated to the appropriate dosages for his/her particular hypertensive condition. After the appropriate dosage of each of the active agents is determined to achieve a decrease of the blood pressure by the predetermined increment without untoward side effects, the patient then may be switched to a single dosage form containing the appropriate dosages of each of the active agents, or may continue with a dual dosage form. Preferably, the single dosage form comprises a first amount of lisinopril from about 2.5 to about 40 mg per day. Preferably, the single dosage form also comprises a second amount of lercanidipine from about 2.5 to about 80 mg per day. In a triple combination, the single dosage form will also comprise from 3.0 to about 50 mg/day, most preferably up to about 25 mg/day HCTZ. In a preferred embodiment, the single dosage form comprises from about 5 to about 20 mg lisinopril and from about 10 to about 20 mg lercanidipine, optionally with about 6-25 mg HCTZ. More preferably, the amount of lisinopril will be 10-20 mg per day. The amount of lercanidipine will be preferably within the range of 2.5-80 mg, more preferably, 10-20 mg. The preferred combinations are: (i) 2.5 mg of lisinopril and 2.5 mg of lercanidipine, (ii) 2.5 mg of lisinopril and 5 mg of lercanidipine, (iii) 2.5 mg lisinopril and 10 mg lercanidipine, (iv) 2.5 mg lisinopril and 20 mg lercanidipine, (v), 2.5 mg of lisinopril and 40 mg lercanidipine, (vi) 2.5 mg of lisinopril and 60 mg lercanidipine, (vii) 2.5 mg of lisinopril and 80 mg lercanidipine, (viii) 5 mg of lisinopril and 2.5 mg of lercanidipine, (ix) 5 mg of lisinopril and 5 mg of lercanidipine, (x) 5 mg of lisinopril and 10 mg of lercanidipine, (xi) 5 mg lisinopril and 20 mg lercanidipine, (xii) 5 mg of lisinopril and 40 mg of lercanidipine, (xiii) 5 mg of lisinopril and 60 mg of lercanidipine, (xiv) 5 mg of lisinopril and 80 mg of lercanidipine, (xv) 10 mg lisinopril and 2.5 mg lercanidipine, (xvi) 10 mg of lisinopril and 5 mg of lercanidipine, (xvii) 10 mg of lisinopril and 10 mg of lercanidipine, (xviii) 10 mg lisinopril and 20 mg lercanidipine, (xix) 10 mg lisinopril and 40 mg lercanidipine, (xx) 10 mg lisinopril and 60 mg lercanidipine, (xxi) 10 mg lisinopril and 80 mg lercanidipine, (xxii) 20 mg lisinopril and 2.5 mg lercanidipine, (xxiii) 20 mg lisinopril and 5 mg lercanidipine, (xxiv) 20 mg lisinopril and 10 mg lercanidipine, (xxv) 20 mg of lisinopril and 20 mg of lercanidipine, (xxvi) 20 mg of lisinopril and 40 mg of lercanidipine, (xxvii) 20 mg of lisinopril and 60 mg of lercanidipine, (xxviii) 20 mg of lisinopril and 80 mg of lercanidipine, (xxix) 40 mg lisinopril and 2.5 mg lercanidipine, (xxx) 40 mg lisinopril and 5 mg lercanidipine, (xxxi) 40 mg lisinopril and 10 mg lercanidipine, (xxxii) 40 mg of lisinopril and 20 mg of lercanidipine, (xxxiii) 40 mg of lisinopril and 40 mg of lercanidipine, (xxxiv) 40 mg of lisinopril and 60 mg of lercanidipine, or (xxxv) 40 mg of lisinopril and 80 mg of lercanidipine.

A pharmaceutical composition for parenteral administration contains from about 0.01% to about 100% by weight of the active agents of the present invention, based upon 100% weight of total pharmaceutical composition.

Generally, transdermal dosage forms contain from about 0.01% to about 100% by weight of the active agents, based upon 100% total weight of the dosage.

The exact dosage and administration regimen utilizing the combination therapy of the present invention is selected in accordance with a variety of factors including type, species, age, weight, sex and medical condition of the patient; the severity and etiology of the hypertension to be treated; the route of administration; the renal and hepatic function of the patient; the treatment history of the patient; and the responsiveness of the patient. Optimal precision in achieving concentrations of active agents within the range that yields efficacy without toxicity requires a regimen based on the kinetics of the drug's availability to target sites. This involves a consideration of the absorption, distribution, metabolism, excretion of a drug, and responsiveness of the patient to the dosage regimen. However, such fine tuning of the therapeutic regimen is routine in light of the guidelines given herein.

The pharmaceutical composition or unit dosage form may be administered in a single daily dose, or the total daily dosage may be administered in divided doses. In addition, co-administration or sequential administration of other active agents may be desirable. For example, addition of a diuretic, a β-receptor blocker, or an angiotensin II receptor antagonist to the combination of lercanidipine and lisinopril is contemplated by the present invention. The dosage amounts of the active agents may be adjusted when combined with other active agents to achieve desired effects (e.g., reduction of blood pressure by a predetermined increment, reduction or avoidance of a particular side-effect).

For combination treatment with both lercanidipine and lisinopril or with all three (i.e., also including HCTZ), where the active agents are in separate dosage formulations, the active agents can be administered concurrently, or they each can be administered at separately staggered times. For example, lisinopril (alone or with diuretic) may be administered in the morning and lercanidipine may be administered in the evening (above or with diuretic depending on the first administration), or vice versa. Additional active agents also may be administered at specific intervals. The order of administration will depend upon the variety of factors including age, weight, sex and medical condition of the patient; the severity and etiology of the hypertension to be treated; the route of administration; the renal and hepatic function of the patient; the treatment history of the patient; and the responsiveness of the patient. Determination of the order of administration may be fine tuned and such fine tuning is routine in light of the guidelines given herein.

In a preferred embodiment of the present invention, the compositions of the invention is administered daily to the patient. In a further embodiment, the composition of lercanidipine and lisinopril (optionally further containing HCTZ) is formulated into a single dosage form.

Patients that may be administered the composition described herein include, without limitation, partial responders or nonresponders to monotherapy with either lisinopril or lercanidipine or with another calcium antagonist or ACE inhibitor and partial responders and nonresponders to other combination therapies. Another class of patients include responders to monotherapy that suffer from dosage-related side-effects, and responders to monotherapy who have been previously determined (or are expected to) become partial responders or nonresponders over time. The classification of patients into nonresponders, partial responders, and responders to a particular antihypertensive regimen is conventionally made by trial and error.

Recently, pharmacogenomic methods involving haplotyping have been utilized to identify responder patients, e.g., U.S. Pat. Nos. 6,200,754; 6,183,958; 6,110,684; and WO 98/45477.

### Uses-Methods for Treating Hypertension

The present invention contemplates a method of treating hypertension by administering to a patient a combination of lercanidipine and lisinopril and optionally including a diuretic, e.g., HCTZ. In preferred embodiments, the combination of the two active agents or three active agents is formulated in one pharmaceutical composition. The patient is administered the combination at prescribed intervals (usually once daily) to maintain a physiologically effective amount of the active agents within the patient's system to produce the desired effect (i.e., a reduction of the patient's blood pressure by the predetermined increment). The composition may be administered by any route, as described above but oral administration is preferred for chronic treatment. The method may be used to treat hypertension in responders, partial responders, and nonresponders of monotherapy.

### EXAMPLES

The present invention will be better understood by reference to the following Examples, which are provided as exemplary of the invention, and not by way of limitation.

### Example I

### Model of Acute Angiotensin-Mediated Renal Hypertension in Anesthetized Rats

### Methods

Male Sprague-Dawley rats, weighing 250-300 g, were anaesthetized with pentobarbital sodium (35 mg/kg, i.p.) and placed on a thermic blanket. The temperature was maintained at 37° C. with a thermoregulator via a rectal probe. The animals were tracheotomized to facilitate spontaneous breathing. A polyethylene catheter was placed in the left jugular vein to allow for infusion of pentobarbital sodium to maintain anesthesia. The left femoral vein and artery were cannulated with polyethylene catheters to allow drugs administration and to monitor blood pressure, respectively.

The animals then underwent a left nephrectomy by excising the left kidney via a flank incision. The right kidney and renal vein, artery and ureter then were exposed via a right retroperitoneal incision, under a dissecting microscope. Silk threads were positioned loosely around both vessels and ureter. The cavity was then covered with Vaseline oil. See Recordati, et al. 2000, J. Hypertension, 18:1277-1287.

After 30-60 minutes of basal recordings of arterial blood pressure and heart rate the threads around the renal vessels and ureter were tied close to the renal hilum to induce complete renal ischemia of the right kidney. After 2 hours of ischemia (designated as “120” in Tables 4-7), the threads were removed to allow renal reperfusion and urine output. The reopening of the renal hilum and restoration of renal circulation, induced an increase in blood pressure that peaked 5-10 minutes and lasted about 60 minutes. Drugs (vehicle, lisinopril (30 μg/kg), lercanidipine (10 μg/kg), or both lercanidipine (10 μg/kg) and lisinopril (30 μg/kg)) were administered intravenously at 5 minutes after reperfusion began (defined as 125 min in Tables 4-7). To evaluate the effects of drugs administration on blood pressure and heart rate, two-way ANOVA and Dunnet's test was used. Statistical analysis was performed on the results obtained at different times during reperfusion, from the administration of the drugs to the end of experiment (125-180 min in the tables). The evaluation of the synergistic effect of combination treatment was carried out by comparing the DBP decrease after 10 minutes from the start of drug administration in the four treatment groups (vehicle, lercanidipine 10 μg/kg, lisinopril 30 μg/kg and the combination treatment), by a factorial model of ANOVA.

### Results

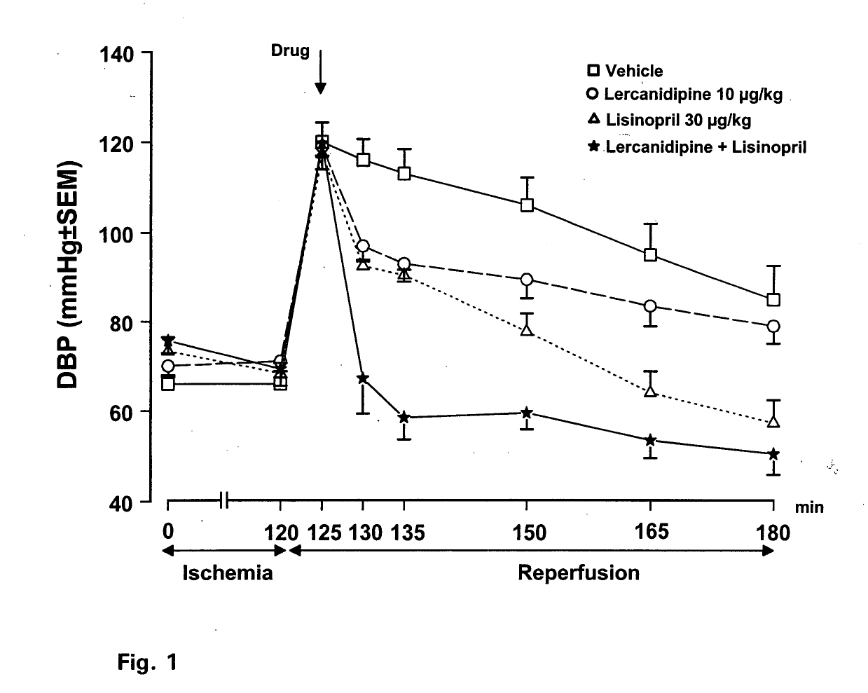

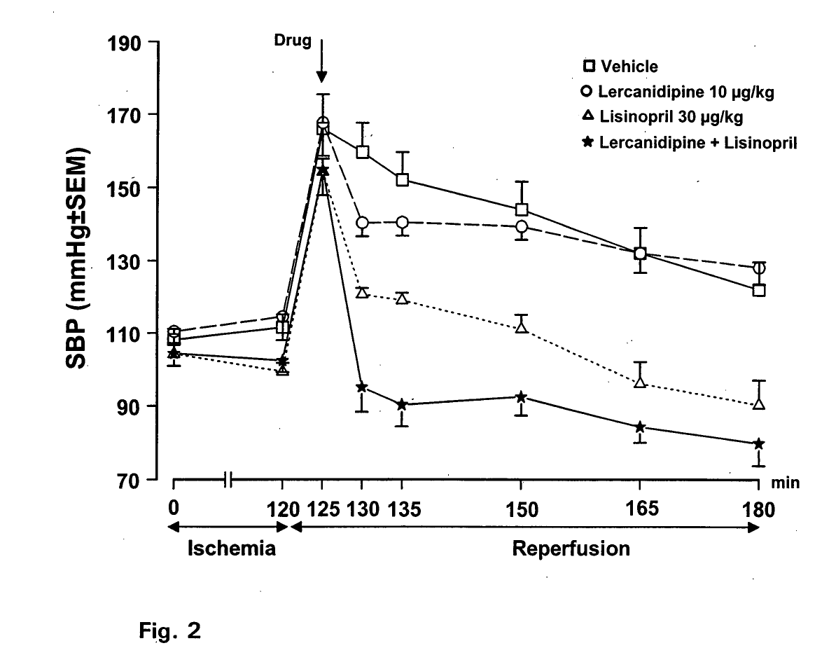

The effects of the different treatments studied are shown in Tables 4-7 and FIG. 1-2.

Administration of either lisinopril or lercanidipine lead to significant decreases in systolic blood pressure and diastolic blood pressure compared to vehicle alone. Combination treatment with lisinopril and lercanidipine lead to significantly greater decreases in both systolic blood pressure and diastolic blood pressure, compared to vehicle along and either administration of lisinopril or lercanidipine alone. The effects on diastolic blood pressure are synergistic, i.e., superadditive, at 10 min after reperfusion (p<0.05) (FIGS. 1 and 2), as shown by the results of statistical analysis (Table 8).

TABLE 4
Time course of the effects on SBP (systolic blood pressure), DBP
(diastolic blood pressure) and HR (heart rate) after intravenous
administration of vehicle (0.5 ml/kg) in uninephrectomized
anesthetized rats. Mean value ± S.E.M. (n = 8).

| TIME | SBP | DBP | HR |
|---|---|---|---|
| min | mmHg | mmHg | beats/min |
| 0 | 108.1 ± 3.1 | 66.0 ± 2.0 | 357.5 ± 14.5 |
| Ischemia | | | |
| 120 | 111.5 ± 3.6 | 65.9 ± 2.8 | 358.1 ± 10.3 |
| Reperfusion | | | |
| 125 | 166.0 ± 9.4 | 119.8 ± 4.3 | 370.4 ± 5.7 |
| Drug | | | |
| 130 | 159.6 ± 8.1 | 115.8 ± 4.6 | 380.6 ± 6.6 |
| 135 | 152.0 ± 7.6 | 112.9 ± 5.4 | 387.3 ± 9.3 |
| 150 | 144.1 ± 7.6^{b} | 106.1 ± 6.2^{b} | 389.0 ± 9.4 |
| 165 | 131.9 ± 6.9^{b} | 94.7 ± 7.0^{b} | 396.9 ± 9.6^{a} |
| 180 | 121.9 ± 7.6^{b} | 84.8 ± 7.6^{b} | 396.9 ± 9.4^{a} |

^{a}= p < 0.05;
^{b}= p < 0.01 vs 125 min (Two way ANOVA and Dunnett's test)

TABLE 5
Time course of the effects on SBP (systolic blood pressure), DBP
(diastolic blood pressure) and HR (heart rate) after intravenous
administration of lisinopril (30 μg/kg) in uninephrectomized
anesthetized rats. Mean value ± S.E.M. (n = 6).

| TIME | SBP | DBP | HR |
|---|---|---|---|
| min | mmHg | mmHg | beats/min |
| 0 | 104.2 ± 3.0 | 73.3 ± 3.2 | 393.3 ± 8.8 |
| Ischemia | | | |
| 120 | 99.3 ± 2.4 | 68.3 ± 2.3 | 390.3 ± 10.0 |
| Reperfusion | | | |
| 125 | 154.3 ± 3.6 | 117.3 ± 1.3 | 398.3 ± 18.8 |
| Drug | | | |
| 130 | 120.7 ± 1.7^{b} | 92.5 ± 1.0^{b} | 408.3 ± 12.3 |
| 135 | 119.0 ± 2.1^{b} | 90.5 ± 1.2^{b} | 405.8 ± 9.7 |
| 150 | 111.0 ± 4.0^{b} | 77.7 ± 4.1^{b} | 406.7 ± 13.1 |
| 165 | 96.0 ± 6.0^{b} | 64.0 ± 4.8^{b} | 406.7 ± 18.2 |
| 180 | 90.2 ± 6.8^{b} | 57.3 ± 5.1^{b} | 400.0 ± 14.6 |

^{b}= p < 0.01 vs 125 min (Two way ANOVA and Dunnett's test)

TABLE 6
Time course of the effects on SBP (systolic blood pressure), DBP
(diastolic blood pressure) and HR (heart rate) after intravenous
administration of lercanidipine (10 μg/kg) in uninephrectomized
anesthetized rats. Mean value ± S.E.M. (n = 8).

| TIME | SBP | DBP | HR |
|---|---|---|---|
| min | mmHg | mmHg | beats/min |
| 0 | 110.4 ± 3.6 | 69.8 ± 2.5 | 359.6 ± 13.9 |
| Ischemia | | | |
| 120 | 114.5 ± 6.5 | 70.6 ± 5.4 | 359.3 ± 7.8 |
| Reperfusion | | | |
| 125 | 167.6 ± 8.9 | 119.3 ± 5.2 | 383.6 ± 9.9 |
| Drug | | | |
| 130 | 140.4 ± 3.8^{b} | 97.1 ± 3.1^{b} | 433.1 ± 8.9^{b} |
| 135 | 140.5 ± 3.7^{b} | 93.1 ± 4.0^{b} | 448.0 ± 6.2^{b} |
| 150 | 139.4 ± 3.7^{b} | 89.5 ± 4.3^{b} | 443.6 ± 8.2^{b} |
| 165 | 132.0 ± 5.3^{b} | 83.5 ± 4.6^{b} | 435.5 ± 11.2^{b} |
| 180 | 128.1 ± 4.7^{b} | 78.8 ± 4.0^{b} | 422.8 ± 12.0^{b} |

^{b}= p < 0.01 vs 125 min (Two way ANOVA and Dunnett's test)

TABLE 7
Time course of the effects on SBP (systolic blood pressure), DBP
(diastolic blood pressure) and HR (heart rate) after intravenous
administration of lercanidipine (10 μg/kg) and lisinopril (30 μg/kg)
in uninephrectomized anesthetized rats.
Mean value ± S.E.M. (n = 6).

| TIME | SBP | DBP | HR |
|---|---|---|---|
| min | mmHg | mmHg | beats/min |
| 0 | 104.3 ± 3.4 | 75.7 ± 2.8 | 397.5 ± 3.6 |
| Ischemia | | | |
| 120 | 102.3 ± 3.2 | 69.3 ± 1.9 | 380.0 ± 11.0 |
| Reperfusion | | | |
| 125 | 154.8 ± 6.9 | 119.7 ± 2.8 | 378.3 ± 6.5 |
| Drug | | | |
| 130 | 95.0 ± 6.6^{b} | 67.2 ± 7.8^{b} | 437.5 ± 10.5^{b} |
| 135 | 90.2 ± 5.8^{b} | 58.5 ± 4.8^{b} | 438.3 ± 9.8^{b} |
| 150 | 92.3 ± 4.9^{b} | 59.5 ± 3.6^{b} | 435.0 ± 6.2^{b} |
| 165 | 84.3 ± 4.3^{b} | 53.5 ± 3.9^{b} | 430.0 ± 8.2^{b} |
| 180 | 79.8 ± 6.1^{b} | 50.5 ± 4.6^{b} | 418.3 ± 4.8^{b} |

^{b}= p < 0.01 vs 125 min (Two way ANOVA and Dunnett's test)

TABLE 8
Mean change of DBP (diastolic blood pressure) after 10 minutes
from the start of drug administration in the four treatment groups
(vehicle, lercanidipine 10 μg/kg, lisinopril 30 μg/kg and the
combination treatment) (Anova factorial model).

| Treatment | DBP mean change ± S.D |
|---|---|
| Vehicle | 6.88 ± 5.00 |
| Lercanidipine | 26.13 ± 8.68^{a} |
| Lisinopril | 26.83 ± 4.40^{a} |
| Lercanidipine + Lisinopril | 61.17 ± 13.92^{b} |

^{a}p < 0.001 compared to treatment with vehicle
^{b}p < 0.05 compared to treatment with lercanidipine alone and lisinopril alone

### Example II

### Association of Lercanidipine and Lisinopril in Renal Hypertensive Dogs

### Methods

Male beagle dogs, 10-12 months old and weighing between 10-11 kg, are used. All dogs are trained for several weeks, to acclimatize them to the test environment. Chronic sustained hypertension is induced in 4 dogs by bilateral renal artery constriction, according to the Goldblatt method “two-kidney, two clip hypertension”. Briefly, under sodium pentobarbital anaesthesia (35 mg/kg i.v.) during two different surgical interventions 15 days apart from each other, in sterile conditions, both renal arteries are clipped with original renal silver clips and narrowed by about 60-70%. After two months from the last intervention, an experimental renal hypertension is produced and the animals are used for the implantation of catheter.

Under sodium pentobarbital anaesthesia (35 mg/kg i.v.), in sterile conditions, the dogs are catheterized by inserting a sensor tipped catheter (Mikro-Tip) into the ascending aorta through the right femoral artery. The catheter is subcutaneously exteriorized at the back of the neck.

After a week recovery time from surgery, the animals are placed in a dog restrained unit, composed by a frame and a dog sling hammock, and connected to the pressure transducers in order to monitor the arterial blood pressure.

All the animals are alternatively treated with vehicle, lercanidipine, lisinopril and the combination of lercanidipine and lisinopril. The drugs are administered by oral gavage in a volume of 1 ml/kg at the following dosages:
(1) Vehicle (1 ml/kg)
(2) lercanidipine (0.5 mg/kg)
(3) lisinopril (3 mg/kg)
(4) lercanidipine+lisinopril (0.5 mg/kg+3 mg/kg)

Blood pressure (systolic and diastolic) is monitored up to 6 h after the administration. Statistical analysis is performed from time 0 to time 360 min. To evaluate the statistical differences among the treatments, data are analyzed using a three-way ANOVA with repeated measures on factor time and pre-planned multiple comparisons. Statistical analysis is performed by means of GLM (general linear model procedure) with SAS software version 6.12.

### Results

Administration of either lisinopril or lercanidipine is tested for significant decreases in systolic blood pressure and/or diastolic blood pressure, compared to vehicle alone. Combination treatment with lisinopril and lercanidipine is tested for greater decreases in systolic blood pressure and/or diastolic blood pressure, compared to vehicle along and/or either administration of lisinopril and lercanidipine alone, i.e., superadditivity.

### Example III

### Clinical Study-Factorial Design

### Methods

Study Design

A multi-center, randomized, double-blind, placebo-controlled, parallel groups, factorial design trial is conducted. After a 14-day (±3 days) screening/washout period during which patients are washed from ongoing antihypertensive therapy, eligible patients with a sitting diastolic blood pressure (SDBP) of 95-109 mm Hg, entered the 28 day (±3 days) single-blind placebo run-in period during which they received placebo once-daily. Patients who are not responsive to this treatment and whose SDBP is 95-109 mm Hg, are randomized into an eight-week double-blind treatment phase. Baseline measurements are taken on Study Day 0 with post-therapy assessments during the double blind period scheduled on Study Days 14, 28, 42, and 56. Laboratory tests are conducted prior to the beginning of the screening period and on study Day 0 as well as at the end of the study.

Number of Patients

A suitable number of patients are screened randomized among the 4-week single-blind placebo period and the 8-week double-blind treatment. Patients are distributed among the treatment grid shown in the following table.

TABLE 9
Patient Distribution Grid

| | | lerc. | lerc. | lerc. | lerc. |
|---|---|---|---|---|---|
| | Placebo | (2.5 mg) | (5 mg) | (10 mg) | (20 mg) |
| Placebo | Group 1 | Group 2 | Group 3 | Group 4 | Group 5 |
| lisinopril | Group 6 | Group 7 | Group 8 | Group 9 | Group 10 |
| (2.5 mg) | | | | | |
| lisinopril | Group 11 | Group 12 | Group 13 | Group 14 | Group 14 |
| (5 mg) | | | | | |
| lisinopril | Group 16 | Group 16 | Group 18 | Group 19 | Group 20 |
| (10 mg) | | | | | |
| lisinopril | Group 21 | Group 22 | Group 23 | Group 24 | Group 25 |
| (20 mg) | | | | | |

Study Drugs and Dosage and Duration of Treatment

Prior histories of patients are taken, to determine response of patients to prior treatment with lercanidipine (lerc.) or lisinopril, or other antihypertensive agents, allowing for determine if patients fall within any of aforementioned groups one through four.

The patients are randomized into one of the above noted 25 treatment groups. Tablets of lercanidipine) and lisinopril are encapsulated in the appropriate doses, in order to assure double-blind conditions. All tablets are of commercial origin.

Patients are instructed to take one capsule of the study medication once daily 15 minutes before breakfast, between 6 and 10 am. All medications are to be withheld on mornings of clinic visits until after blood pressure evaluations at 22-26 hours following the previous day's dose of study medication.

Parameters Evaluated

Efficacy:

1. Trough (Minimum) (24±2 hours post-dose) sitting diastolic blood pressure (SDBP)
2. Trough (24±2 hours post-dose) sitting systolic blood pressure (SSBP)
3. Standing DBP and standing SBP—immediate and after 2 minutes
4. Percent of patients with SDBP <90 mm Hg
5. Percent of patients with SDBP <90 mm Hg or with SDBP ≧90 mm Hg but a decrease of ≧10 mm Hg
6. Percent of patients with SDBP <85 mm Hg
   Safety:
1. Adverse events
2. Electrocardiogram
3. Laboratory tests
4. Physical exam
5. Heart rate
6. Changes in DBP and SDP from sitting to standing
   Statistical Methods

The primary efficacy variable is the change from baseline to mean trough SDBP after 8 weeks of double-blind treatment. The analysis is performed in an “intent-to-treat” fashion, including all randomized patients who receive at least one dose of double-blind treatment, and at least one post-baseline SDBP measurement 18-48 hours post-dose. In case of premature withdrawal from the study the last observation carried forward (LOCF) algorithm is applied (“end point analysis”).

The overall treatment comparison is performed using an analysis of covariance (ANCOVA) with treatment and centers as main effects and baseline value as covariate.

An additional ANOVA is performed using the factorial model in order to test for the interaction of lercanidipine dose and lisinopril dose at each time-point.

Results of patients treated with either lisinopril or lercanidipine composition exhibit are analyzed to determine if significant decrease in blood pressure compared to patients that are given placebo has occurred. Results of patients treated with a combination composition of lercanidipine and lisinopril are tested for effectiveness greater than treatment with either agent alone.

Therapeutic Response Rates (SDBP <90 mm Hg)

The response rates (SDBP decreased below 90 mm Hg) for the patients within each dosage group are determined. It should be noted that achievement of SDBP <90 mm Hg is not an aim of this study.

### Example IV

### Effect of Lercanidipine on Lisinopril Treatment

### Methods

Study Design

This is a multicenter, randomized, double-blind, parallel-group study of the efficacy and safety of lercanidipine versus a comparative agent as an add-on therapy in patients with mild to moderate essential hypertension, uncontrolled on previous lisinopril treatment.

The study is divided into three periods:
(1) During a lead-in period, naive patients are administered a stable dose of lisinopril for 4 weeks. For patients already on a stable dose of lisinopril for at least 2 weeks prior to study entry, the lead-in period is reduced to 2 weeks.
(2) The lead-in period is followed by a 2-week single-blind, run-in period where placebo is added to the current lisinopril therapy to establish a baseline SDBP under stable lisinopril therapy.
(3) After the run-in period, patients with a SDBP between 90 and 109 mm Hg and a SSBP between 140 and 179 mm Hg, inclusive, are administered 10 mg; leranidipine as add-on therapy for 8 weeks. For patients whose SBDP is ≧85 mm Hg after 4 weeks of treatment, the dose of lercanidipine is increased to 20 mg.
   Study Drugs and Dosage and Duration of Treatment

Patients were instructed to take medication once daily.

Parameters Evaluated

Efficacy:

1. Trough (Minimum) (24±2 hours post-dose) SDBP
2. Trough (24±2 hours post-dose) SSBP
3. Percent of patients with SDBP <90 mm Hg after 4 and 8 weeks of treatment
4. Percent of patients with BP <140/90 mm Hg after 4 and 8 weeks of treatment

### Results

Results are analyzed for indication that addition of lercanidipine to existing lisinopril therapy decreases SSBP greater than would be suggested when lisinopril and lercanidipine are administered as monotherapies. Studies are analyzed for indication that the effect associated with the combination of lisinopril and lercanidipine is greater than the anti-hypertensive effect produced by the conventional dosages of either lercanidipine alone and lisinopril alone.

Studies are also analyzed for indication that treatment with combination treatment with sub-threshold amounts of each of lisinopril and lercanidipine leads to normalization of blood pressure in patients with essential hypertension.

### Example V

### Effect of Lisinopril on Lercanidipine Treatment

The inverse of the study of Example IV is performed to determine the effect of addition of lisinopril to lercanidipine monotherapy.

Results are analyzed for indication that addition of lisinopril to existing lercanidipine therapy decreases SSBP greater than would be suggested when lisinopril and lercanidipine are administered as monotherapies. Studies are analyzed for indication that the effect associated with the combination of lisinopril and lercanidipine is greater than the anti-hypertensive effect produced by the conventional dosages of either lercanidipine alone and lisinopril alone.

Studies are also analyzed for indication that treatment with combination treatment with sub-threshold amounts of each of lisinopril and lercanidipine leads to normalization of blood pressure in patients with essential hypertension.

### Example VI

### Model of Acute Angiotensin-Mediated Renal Hypertension in Anesthetized Rats

### Methods

Male Sprague-Dawley rats, weighing 250-300 g, were anaesthetized with pentobarbital sodium (35 mg/kg, i.p.) and placed on a thermic blanket. The temperature was maintained at 37° C. with a thermoregulator via a rectal probe. The animals were tracheotomized to facilitate spontaneous breathing. A polyethylene catheter was placed in the left jugular vein to allow for infusion of pentobarbital sodium to maintain anesthesia. The left femoral vein and artery were cannulated with polyethylene catheters to allow drugs administration and to monitor blood pressure, respectively.

Animals then underwent a left nephrectomy by excising the left kidney via a flank incision. The right kidney and renal vein, artery and ureter were then exposed via a right retroperitoneal incision, under a dissecting microscope. Silk threads were placed around both vessels and ureter. The cavity was then covered with Vaseline oil. See Recordati, et al. 2000, J. Hypertension, 18:1277-1287.

After 30-60 minutes of basal recordings of arterial blood pressure and heart rate the threads around the renal vessels and ureter were tied close to the renal hilum to induce complete renal ischemia of the right kidney. After 2 hours of ischemia, the threads were removed to allow renal reperfusion and urine output. The reopening of the renal hilum and restoration of renal circulation, induced an increase in blood pressure that peaked at 5-10 min and lasted about 60 min. The following drugs were administered intravenously 5 minutes after reperfusion was begun:
vehicle (VE) 1 ml/kg,
lisinopril+hydrochlorothiazide (LIS+HCTZ) 10+12 μg/kg,
lercanidipine (LER) 7.5 μg/kg,
combination of lisinopril+hydrochlorothiazide and lercanidipine (COMBO) 10+12+7.5 μg/kg.

Results are expressed as mean values ±S.E in the tables and as mean values ±S.E. and Δ±S.E. changes in the figures.

To evaluate the effects of drugs administration on blood pressure within each group (“within treatment”), the data were analysed using a three-way ANOVA with repeated measures on factor time and pre-planned multiple comparisons, using SAS software version 8.2.

To evaluate the additive or synergistic effect of the drugs administered, the Δ values (root square transformation) of the groups LIS+HCTZ, LER and COMBO at the times 5, 10 and 25 min after the administration were compared by means of a ANOVA, using SAS software version 8.2.

### Results

Effects of Treatments on BP

Following reperfusion, a marked and fast increase of blood pressure, that had its peak at 5 min after the reopening of vascular renal hilum, was observed (Table 10-13). In order to compare the effects of single drugs and their combination on the reperfusion-induced hypertension, the compounds were intravenously administered at the peak effect of hypertension (5 min) and statistical analysis (“within groups”) was performed from the administration time to the end of experiment (time 125-180 min in the tables and figures).

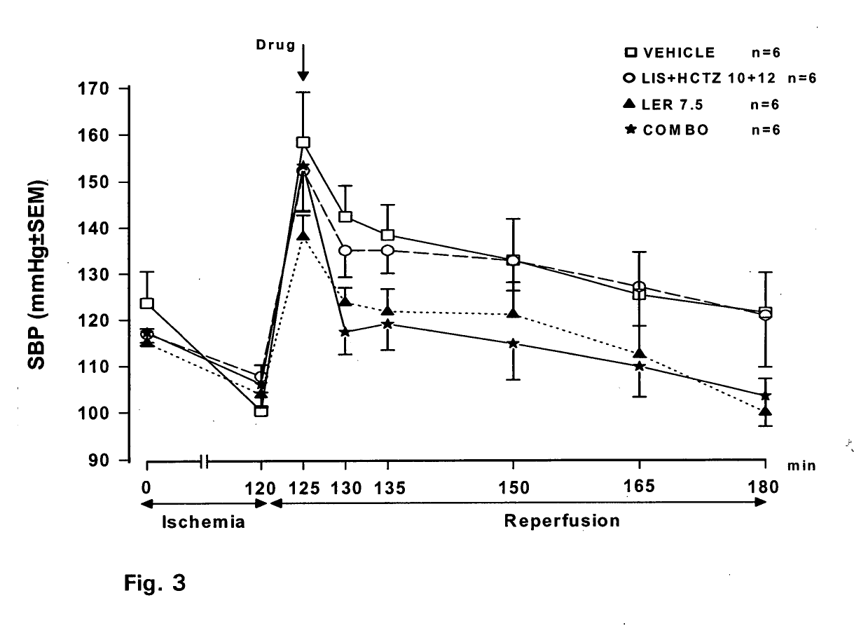

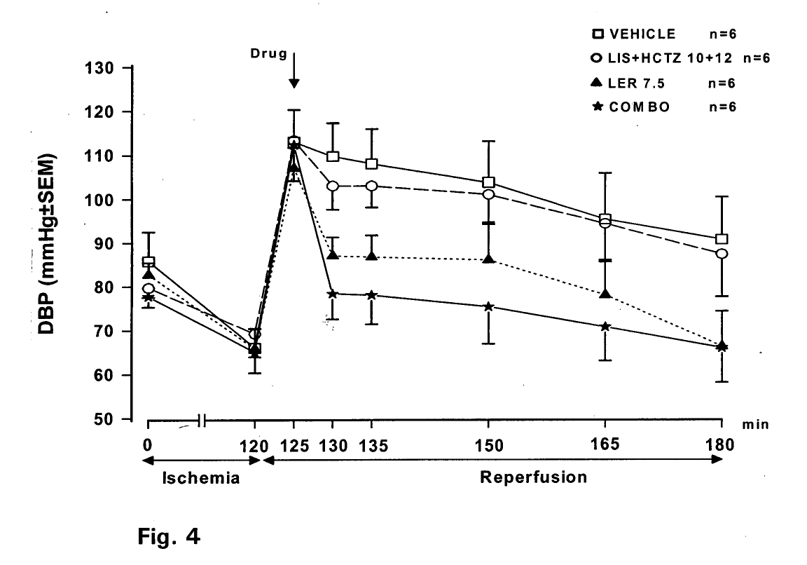

In the vehicle-treated group, the hypertensive state was maintained without statistically significant decrease in diastolic blood pressure up to 40 min after the kidney reperfusion, (Table 10; FIGS. 3 and 4); the administration of lisinopril+hydrochlorothiazide (10+12 μg/kg) induced a slight decrease in blood pressure, statistically significant on systolic blood pressure from 25 to 55 min, and on diastolic form 40 to 55 min after administration (Table 11; FIGS. 3 and 4).

Lercanidipine at 7.5 μg/kg, induced a statistically significant decrease in diastolic blood pressure from 5 to 55 min after administration, whereas the effects on systolic blood pressure were statistically significant at 40 and 55 after administration (Table 12; FIGS. 3 and 4). The antihypertensive effect of lercanidipine was higher than that induced by lisinopril+hydrochlorothiazide.

When lercanidipine and lisinopril+hydrochlorothiazide were administered togheter (7.5 μg/kg+10+12 μg/kg, respectively) they induced a rapid and significant fall in blood pressure, reversing immediately the ischemia-induced hypertension and lowering blood pressure to normotensive pre-ischemic levels with statistically significant effect on both systolic and diastolic blood pressure from 5 to 55 min (p<0.01) (Table 13; FIGS. 3 and 4).

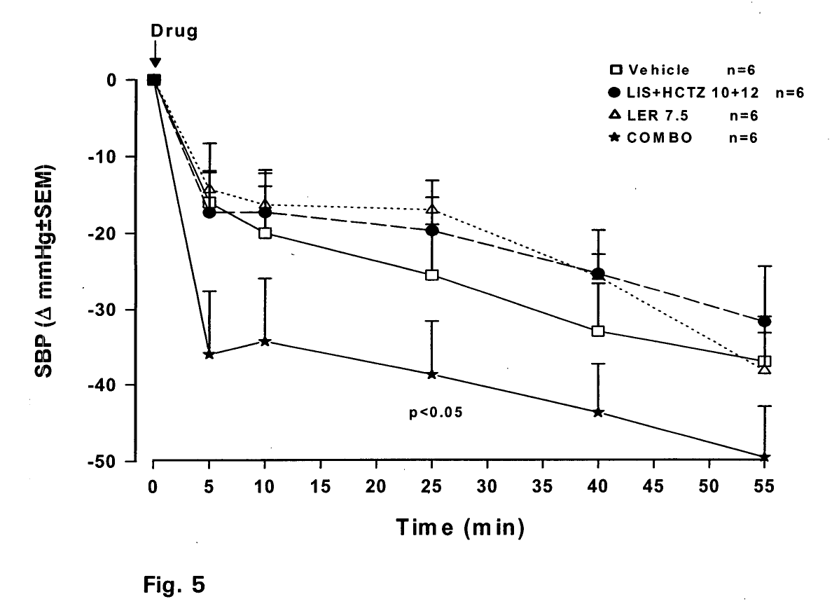

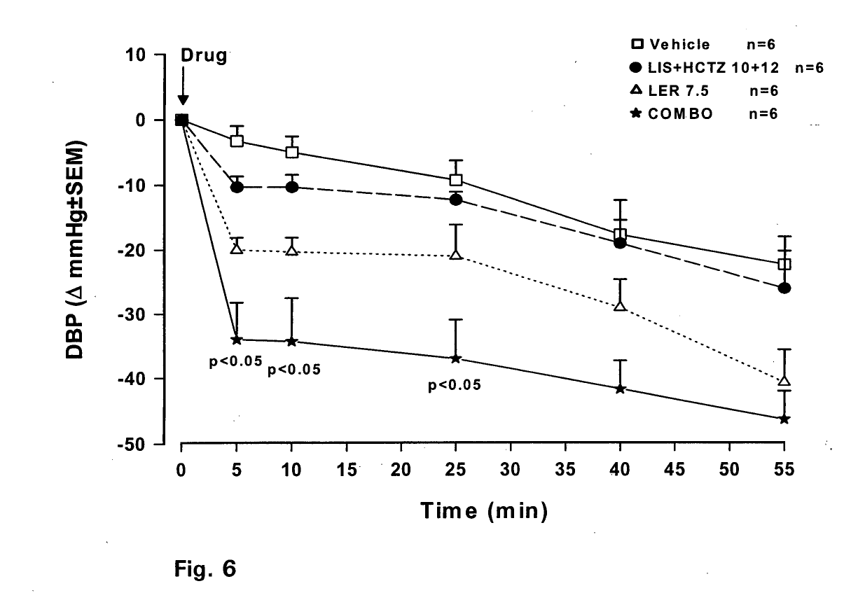

In order to evaluate whether the administration of COMBO induced additive or synergistic antihypertensive effect, statistical analysis was performed on A SBP and A DBP values, as described in the Methods Section. At 5, 10 and 25 min after the administration the decrease in systolic and diastolic blood pressure was higher than the sum of the decrease induced by each single drug. Thus, the combination resulted in an unexpected augmented decrease in diastolic blood pressure (FIG. 5). The effect of combination treatment on systolic blood pressure was additive at 5 and 10 min and augmented at 25 min after the administration (FIG. 6).

Effects of Treatments on HR

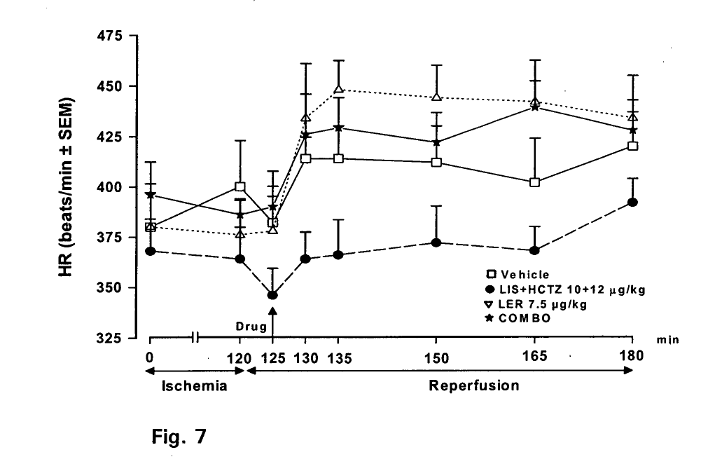

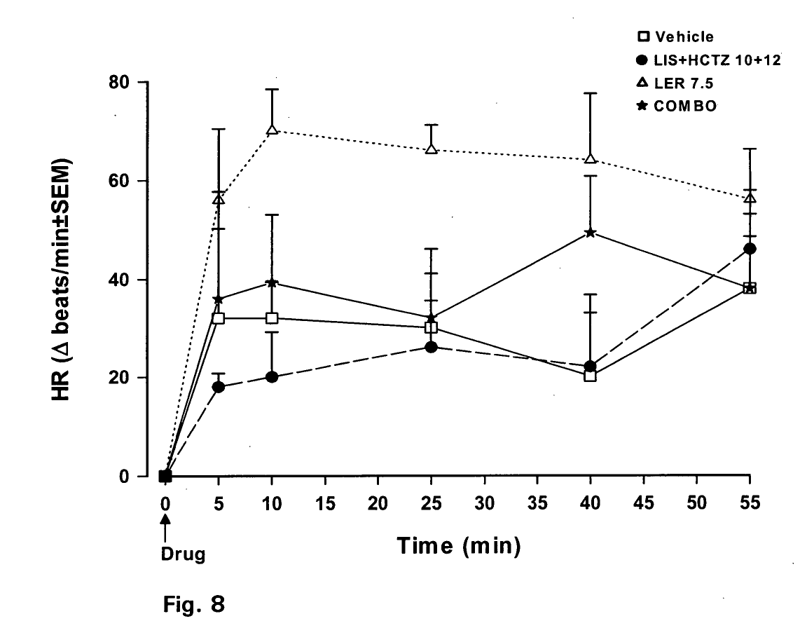

Vehicle and lisinopril+hydrochlorothiazide induced a slight, non-statistically significant increases on heart rate (Tables 10 and 11 and FIGS. 7 and 8), whereas lercanidipine was characterized by a positive chronotropic effect, probably due to the baroreflex-activation, that was more marked than that observed in the vehicle group (Table 12 and FIGS. 7 and 8).

When lercanidipine was administered together with lisinopril+hydrochlorothiazide the changes in heart rate were not different from those of the vehicle- and lisinopril+hydrochlorothiazide-treated groups (Table 13 and FIGS. 7 and 8): the combination of lercanidipine and lisinopril+hydrochlorothiazide reduced the tachycardic effect due to acute administration of lercanidipine alone.

TABLE 10
Time course of the effects on SBP (systolic blood pressure), DBP
(diastolic blood pressure) and HR (heart rate) after intravenous
administration of vehicle (1 ml/kg) in uninephrectomized
anesthetized rats. Mean value ± S.E.M. (n = 6).

| Time | SBP | DBP | HR |
|---|---|---|---|
| Min | mmHg | mmHg | beats/min |
| 0 | 123.7 ± 6.8 | 85.8 ± 6.7 | 380.0 ± 21.6 |
| Ischemia | | | |
| 120 | 100.3 ± 4.0 | 66.0 ± 4.3 | 400.0 ± 22.9 |
| Reperfusion | | | |
| 125 | 158.3 ± 10.7 | 113.0 ± 7.2 | 382.0 ± 18.2 |
| Drug | | | |
| 130 | 142.3 ± 6.7 | 109.7 ± 7.5 | 414.0 ± 10.6 |
| 135 | 138.3 ± 6.5 a | 108.0 ± 7.9 | 414.0 ± 16.3 |
| 150 | 132.8 ± 8.9 b | 103.7 ± 9.4 | 412.0 ± 18.2 |
| 165 | 125.3 ± 9.2 b | 95.3 ± 10.5 a | 402.0 ± 22.1 |
| 180 | 121.3 ± 8.7 b | 90.7 ± 9.7 b | 420.0 ± 17.0 |

a = p < 0.05;
b = p < 0.01 vs 125 min (within treatment)

TABLE 11
Time course of the effects on SBP (systolic blood pressure). DBP
(diastolic blood pressure) and HR (heart rate) after intravenous
administration of lisinopril+ hydrochlorothiazide
(10 + 12 μg/kg) in uninephrectomized
anesthetized rats. Mean value ± S.E.M. (n = 6).

| Time | SBP | DBP | HR |
|---|---|---|---|
| min | mmHg | mmHg | beats/min |
| 0 | 117.0 ± 2.5 | 79.7 ± 2.2 | 368.0 ± 16.0 |
| Ischemia | | | |
| 120 | 107.7 ± 6.8 | 69.2 ± 5.2 | 364.0 ± 15.7 |
| Reperfusion | | | |
| 125 | 152.3 ± 8.7 | 113.3 ± 6.4 | 346.0 ± 13.3 |
| Drug | | | |
| 130 | 135.0 ± 5.8 | 103.0 ± 5.4 | 364.0 ± 13.4 |
| 135 | 135.0 ± 5.0 | 103.0 ± 4.9 | 366.0 ± 17.5 |
| 150 | 132.7 ± 6.5 a | 101.0 ± 6.4 | 372.0 ± 18.3 |
| 165 | 127.0 ± 8.6 b | 94.3 ± 8.3 b | 368.0 ± 11.9 |
| 180 | 120.7 ± 11.2 b | 87.3 ± 9.7 b | 392.0 ± 11.9 |

a = p < 0.05;
b = p < 0.01 vs 125 min (within treatment).

TABLE 12
Time course of the effects on SBP (systolic blood pressure). DBP
(diastolic blood pressure) and HR (heart rate) after intravenous
administration of lercanidipine (7.5 μg/kg) in uninephrectomized
anesthetized rats. Mean value ± S.E.M. (n = 6).

| Time | SBP | DBP | HR |
|---|---|---|---|
| min | mmHg | mmHg | beats/min |
| 0 | 115.0 ± 3.1 | 82.7 ± 2.7 | 380.0 ± 17.2 |
| Ischemia | | | |
| 120 | 103.8 ± 6.4 | 65.8 ± 4.6 | 376.0 ± 17.0 |
| Reperfusion | | | |
| 125 | 138.0 ± 4.6 | 107.0 ± 4.0 | 378.0 ± 17.2 |
| Drug | | | |
| 130 | 123.7 ± 3.2 | 87.0 ± 4.3 b | 434.0 ± 26.9 b |
| 135 | 121.7 ± 4.9 | 86.7 ± 5.0 b | 448.0 ± 14.4 b |
| 150 | 121.0 ± 7.0 | 86.0 ± 8.2 b | 444.0 ± 16.1 b |
| 165 | 112.3 ± 6.1 b | 78.0 ± 7.6 b | 442.0 ± 20.4 b |
| 180 | 99.8 ± 7.2 b | 66.3 ± 8.0 b | 434.0 ± 20.9 b |

b = p < 0.01 vs 125 min (within treatment).

TABLE 13
Time course of the effects on SBP (systolic blood pressure). DBP
(diastolic blood pressure) and HR (heart rate) after intravenous
administration of COMBO: lisinopril+ hydrochlorothiazide
(10 + 12 μg/kg) and lercanidipine (7.5 μg/kg) in
uninephrectomized anesthetized rats. Mean value ± S.E.M.
(n = 6).

| Time | SBP | DBP | HR |
|---|---|---|---|
| min | mmHg | mmHg | beats/min |
| 0 | 117.3 ± 2.2 | 77.7 ± 2.4 | 396.0 ± 16.4 |
| Ischemia | | | |
| 120 | 106.0 ± 4.7 | 65.0 ± 4.7 | 386.0 ± 7.8 |
| Reperfusion | | | |
| 125 | 153.3 ± 9.9 | 112.3 ± 8.2 | 390.0 ± 17.7 |
| Drug | | | |
| 130 | 117.3 ± 4.9 b | 78.3 ± 5.8 b | 426.0 ± 19.8 |
| 135 | 119.0 ± 5.7 b | 78.0 ± 6.6 b | 429.3 ± 14.9 |
| 150 | 114.7 ± 7.9 b | 75.3 ± 8.4 b | 422.0 ± 14.7 |
| 165 | 109.7 ± 6.6 b | 70.7 ± 7.6 b | 439.3 ± 13.0 a |
| 180 | 103.3 ± 6.5 b | 66.0 ± 7.9 b | 428.0 ± 14.8 |

a = p < 0.05;
b = p < 0.01 vs 125 min (within treatment).

The present invention is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims.

It is further to be understood that values are approximate, and are provided for description.

Patents, patent applications, publications, procedures, and the like are cited throughout this application, the disclosures of which are incorporated herein by reference in their entireties.

1. A method for treating hypertension in a patient in need thereof, said method comprising administering to said patient a first amount of lercanidipine and a second amount of lisinopril, wherein said amounts in combination are effective to reduce blood pressure in said patient by at least a predetermined increment; wherein each of said first amount and said second amount administered individually is ineffective to produce a reduction in blood pressure in said patient or said reduction in blood pressure is less than said predetermined increment.

2. The method as defined in claim 1, wherein said first amount is from about 2.5-80 mg per day.

3. The method as defined in claim 2, wherein said first amount is from about 10-20 mg per day.

4. The method as defined in claim 3, wherein said second amount is from about 2.5-40 mg per day.

5. The method as defined in claim 4, wherein said second amount is from about 5-20 mg per day.

6. The method as defined in claim 1, wherein (i) said first amount is 2.5 mg per day and said second amount is 2.5 mg per day, (ii) said first amount is 2.5 mg per day and said second amount is 5 mg per day, (iii) said first amount is 2.5 mg per day and said second amount is 10 mg per day, (iv) said first amount is 2.5 mg per day and said second amount is 20 mg per day, (v) said first amount is 2.5 mg per day and said second amount is 40 mg per day, (vi) said first amount is 5 mg per day and said second amount is 2.5 mg per day, (vii) said first amount is 5 mg per day and said second amount is 0.0 mg per day, (viii) said first amount is 5 mg per day and said second amount is 10 mg per ay, (ix) said first amount is 5 mg per day and said second amount is 20 mg per day, (x) said first amount is 5 mg per day and said second amount is 40 mg per day, (xi) said first amount is 10 mg per day and said second amount is 2.5 mg per day, (xii) said first amount is 10 mg per day and said second amount is 5 mg per day, (xiii) said first amount is 10 mg per day and said second amount is 10 mg per day, (xiv) said first amount is 10 mg per day and said second amount is 20 mg per day, (xv) said first amount is 10 mg per day and said second amount is 40 mg per day, (xvi) said first amount is 20 mg per day and said second amount is 2.5 mg per day, (xvii) said first amount is 20 mg per day and said second amount is 5 mg per day, (xviii) said first amount is 20 mg per day and said second amount is 10 mg per day, (xix) said first amount is 20 mg per day and said second amount is 20 mg per day, (xx) said first amount is 20 mg per day and said second amount is 40 mg per day, (xxi) said first amount is 40 mg per day and said second amount is 2.5 mg per day, (xxii) said first amount is 40 mg per day and said second amount is 5 mg per day, (xxiii) said first amount is 40 mg per day and said second amount is 10 mg per day, (xxiv) said first amount is 40 mg per day and said second amount is 20 mg per day, (xxv) said first amount is 40 mg per day and said second amount is 40 mg per day, (xxvi) said first amount is 60 mg per day and said second amount is 2.5 mg per day, (xxvii) said first amount is 60 mg per day and said second amount is 5 mg per day, (xxviii) said first amount is 60 mg per day and said second amount is 10 mg per day, (xxix) said first amount is 60 mg per day and said second amount is 20 mg per day, (xxx) said first amount is 60 mg per day and said second amount is 40 mg per day, (xxxi) said first amount is 80 mg per day and said second amount is 2.5 mg per day, (xxxii) said first amount is 80 mg per day and said second amount is 5 mg per day, (xxxiii) said first amount is 80 mg per day and said second amount is 10 mg per day, (xxxiv) said first amount is 80 mg per day and said second amount is 20 mg per day, or (xxxv) said first amount is 80 mg per day and said second amount is 40 mg per day.

7. A method for treating hypertension in a nonresponder patient in need thereof, said method comprising administering to said patient a first amount of lercanidipine and a second amount of lisinopril, wherein said amounts in combination are effective to reduce blood pressure in said patient by at least a predetermined increment, wherein said patient has been previously determined to be a nonresponder to monotherapy with lercanidipine or a nonresponder to combination therapy involving a different combination of antihypertensive agents.

8. The method as defined in claim 7, wherein said first amount is from about 2.5-80 mg per day.

9. The method as defined in claim 8, wherein said first amount is from about 10-20 mg per day.

10. The method as defined in claim 9, wherein said second amount is from about 2.5-40 mg per day.

11. The method as defined in claim 10, wherein said second amount is from about 5-20 mg per day.

12. The method as defined in claim 7, wherein (i) said first amount is 2.5 mg per day and said second amount is 2.5 mg per day, (ii) said first amount is 2.5 mg per day and said second amount is 5 mg per day, (iii) said first amount is 2.5 mg per day and said second amount is 10 mg per day, (iv) said first amount is 2.5 mg per day and said second amount is 20 mg per day, (v) said first amount is 2.5 mg per day and said second amount is 40 mg per day, (vi) said first amount is 5 mg per day and said second amount is 2.5 mg per day, (vii) said first amount is 5 mg per day and said second amount is 5 mg per day, (viii) said first amount is 5 mg per day and said second amount is 10 mg per ay, (ix) said first amount is 5 mg per day and said second amount is 20 mg per day, (x) said first amount is 5 mg per day and said second amount is 40 mg per day, (xi) said first amount is 10 mg per day and said second amount is 2.5 mg per day, (xii) said first amount is 10 mg per day and said second amount is 5 mg per day, (xiii) said first amount is 10 mg per day and said second amount is 10 mg per day, (xiv) said first amount is 10 mg per day and said second amount is 20 mg per day, (xv) said first amount is 10 mg per day and said second amount is 40 mg per day, (xvi) said first amount is 20 mg per day and said second amount is 2.5 mg per day, (xvii) said first amount is 20 mg per day and said second amount is 5 mg per day, (xviii) said first amount is 20 mg per day and said second amount is 10 mg per day, (xix) said first amount is 20 mg per day and said second amount is 20 mg per day, (xx) said first amount is 20 mg per day and said second amount is 40 mg per day, (xxi) said first amount is 40 mg per day and said second amount is 2.5 mg per day, (xxii) said first amount is 40 mg per day and said second amount is 5 mg per day, (xxiii) said first amount is 40 mg per day and said second amount is 10 mg per day, (xxiv) said first amount is 40 mg per day and said second amount is 20 mg per day, (xxv) said first amount is 40 mg per day and said second amount is 40 mg per day, (xxvi) said first amount is 60 mg per day and said second amount is 2.5 mg per day, (xxvii) said first amount is 60 mg per day and said second amount is 5 mg per day, (xxviii) said first amount is 60 mg per day and said second amount is 10 mg per day, (xxix) said first amount is 60 mg per day and said second amount is 20 mg per day, (xxx) said first amount is 60 mg per day and said second amount is 40 mg per day, (xxxi) said first amount is 80 mg per day and said second amount is 2.5 mg per day, (xxxii) said first amount is 80 mg per day and said second amount is 5 mg per day, (xxxiii) said first amount is 80 mg per day and said second amount is 10 mg per day, (xxxiv) said first amount is 80 mg per day and said second amount is 20 mg per day, or (xxxv) said first amount is 80 mg per day and said second amount is 40 mg per day.

13. A method for treating hypertension in a partial responder patient in need thereof, said method comprising administering to said patient a first amount of lercanidipine and a second amount of lisinopril, wherein said amounts in combination are effective to reduce blood pressure in said patient by at least a predetermined increment, wherein each of said first amount and said second amount administered individually is ineffective to produce a reduction in blood pressure by said predetermined increment.

14. The method as defined in claim 13, wherein said first amount is from about 2.5-80 mg per day.

15. The method as defined in claim 13, wherein said first amount is from about 10-20 mg per day.

16. The method as defined in claim 15, wherein said second amount is from about 2.5-40 mg per day.

17. The method as defined in claim 16, wherein said second amount is from about 5-20 mg per day.

18. The method as defined in claim 13, wherein (i) said first amount is 2.5 mg per day and said second amount is 2.5 mg per day, (ii) said first amount is 2.5 mg per day and said second amount is 5 mg per day, (iii) said first amount is 2.5 mg per day and said second amount is 10 mg per day, (iv) said first amount is 2.5 mg per day and said second amount is 20 mg per day, (v) said first amount is 2.5 mg per day and said second amount is 40 mg per day, (vi) said first amount is 5 mg per day and said second amount is 2.5 mg per day, (vii) said first amount is 5 mg per day and said second amount is 5 mg per day, (viii) said first amount is 5 mg per day and said second amount is 10 mg per ay, (ix) said first amount is 5 mg per day and said second amount is 20 mg per day, (x) said first amount is 5 mg per day and said second amount is 40 mg per day, (xi) said first amount is 10 mg per day and said second amount is 2.5 mg per day, (xii) said first amount is 10 mg per day and said second amount is 5 mg per day, (xiii) said first amount is 10 mg per day and said second amount is 10 mg per day, (xiv) said first amount is 10 mg per day and said second amount is 20 mg per day, (xv) said first amount is 10 mg per day and said second amount is 40 mg per day, (xvi) said first amount is 20 mg per day and said second amount is 2.5 mg per day, (xvii) said first amount is 20 mg per day and said second amount is 5 mg per day, (xviii) said first amount is 20 mg per day and said second amount is 10 mg per day, (xix) said first amount is 20 mg per day and said second amount is 20 mg per day, (xx) said first amount is 20 mg per day and said second amount is 40 mg per day, (xxi) said first amount is 40 mg per day and said second amount is 2.5 mg per day, (xxii) said first amount is 40 mg per day and said second amount is 5 mg per day, (xxiii) said first amount is 40 mg per day and said second amount is 10 mg per day, (xxiv) said first amount is 40 mg per day and said second amount is 20 mg per day, (xxv) said first amount is 40 mg per day and said second amount is 40 mg per day, (xxvi) said first amount is 60 mg per day and said second amount is 2.5 mg per day, (xxvii) said first amount is 60 mg per day and said second amount is 5 mg per day, (xxviii) said first amount is 60 mg per day and said second amount is 10 mg per day, (xxix) said first amount is 60 mg per day and said second amount is 20 mg per day, (xxx) said first amount is 60 mg per day and said second amount is 40 mg per day, (xxxi) said first amount is 80 mg per day and said second amount is 2.5 mg per day, (xxxii) said first amount is 80 mg per day and said second amount is 5 mg per day, (xxxiii) said first amount is 80 mg per day and said second amount is 10 mg per day, (xxxiv) said first amount is 80 mg per day and said second amount is 20 mg per day, or (xxxv) said first amount is 80 mg per day and said second amount is 40 mg per day.

19. A method for treating hypertension in a patient in need thereof, wherein said patient has been previously determined to be responsive to at least one of lercanidipine monotherapy or lisinopril monotherapy but has ceased being adequately responsive thereto said method comprising administering to said patient a first amount of lercanidipine and a second amount of lisinopril in combination, wherein said first amount and said second amount are in combination effective to reduce said patient's blood pressure by at least a predetermined increment.

20. The method as defined in claim 19, wherein said first amount is from about 2.5-80 mg per day.

21. The method as defined in claim 20, wherein said first amount is from about 10-20 mg per day.

22. The method as defined in claim 21, wherein said second amount is from about 2.5-40 mg per day.

23. The method as defined in claim 22, wherein said second amount is from about 5-20 mg per day.

24. The method as defined in claim 19, wherein (i) said first amount is 2.5 mg per day and said second amount is 2.5 mg per day, (ii) said first amount is 2.5 mg per day and said second amount is 5 mg per day, (iii) said first amount is 2.5 mg per day and said second amount is 10 mg per day, (iv) said first amount is 2.5 mg per day and said second amount is 20 mg per day, (v) said first amount is 2.5 mg per day and said second amount is 40 mg per day, (vi) said first amount is 5 mg per day and said second amount is 2.5 mg per day, (vii) said first amount is 5 mg per day and said second amount is 5 mg per day, (viii) said first amount is 5 mg per day and said second amount is 10 mg per ay, (ix) said first amount is 5 mg per day and said second amount is 20 mg per day, (x) said first amount is 5 mg per day and said second amount is 40 mg per day, (xi) said first amount is 10 mg per day and said second amount is 2.5 mg per day, (xii) said first amount is 10 mg per day and said second amount is 5 mg per day, (xiii) said first amount is 10 mg per day and said second amount is 10 mg per day, (xiv) said first amount is 10 mg per day and said second amount is 20 mg per day, (xv) said first amount is 10 mg per day and said second amount is 40 mg per day, (xvi) said first amount is 20 mg per day and said second amount is 2.5 mg per day, (xvii) said first amount is 20 mg per day and said second amount is 5 mg per day, (xviii) said first amount is 20 mg per day and said second amount is 10 mg per day, (xix) said first amount is 20 mg per day and said second amount is 20 mg per day, (xx) said first amount is 20 mg per day and said second amount is 40 mg per day, (xxi) said first amount is 40 mg per day and said second amount is 2.5 mg per day, (xxii) said first amount is 40 mg per day and said second-amount is 5 mg per day, (xxiii) said first amount is 40 mg per day and said second amount is 10 mg per day, (xxiv) said first amount is 40 mg per day and said second amount is 20 mg per day, (xxv) said first amount is 40 mg per day and said second amount is 40 mg per day, (xxvi) said first amount is 60 mg per day and said second amount is 2.5 mg per day, (xxvii) said first amount is 60 mg per day and said second amount is 5 mg per day, (xxviii) said first amount is 60 mg per day and said second amount is 10 mg per day, (xxix) said first amount is 60 mg per day and said second amount is 20 mg per day, (xxx) said first amount is 60 mg per day and said second amount is 40 mg per day, (xxxi) said first amount is 80 mg per day and said second amount is 2.5 mg per day, (xxxii) said first amount is 80 mg per day and said second amount is 5 mg per day, (xxxiii) said first amount is 80 mg per day and said second amount is 10 mg per day, (xxxiv) said first amount is 80 mg per day and said second amount is 20 mg per day, or (xxxv) said first amount is 80 mg per day and said second amount is 40 mg per day.

25. The method as defined in claim 1, wherein said lisinopril and said lercanidipine are administered to said patient in a single dosage form.

26. The method as defined in claim 7, wherein said lisinopril and said lercanidipine are administered to said patient in a single dosage form.

27. The method as defined in claim 13, wherein said lisinopril and said lercanidipine are administered to said patient in a single dosage form.

28. The method as defined in claim 19, wherein said lisinopril and said lercanidipine are administered to said patient in a single dosage form.

29. The method as defined in claim 1, wherein said lisinopril and said lercanidipine are present in a composition.

30. The method as defined in claim 7, wherein said lisinopril and said lercanidipine are present in a composition.

31. The method as defined in claim 13, wherein said lisinopril and said lercanidipine are present in a composition.

32. The method as defined in claim 19, wherein said lisinopril and said lercanidipine are present in a composition.

33. A pharmaceutical composition for treatment of hypertension comprising a first amount of lercanidipine and a second amount of lisinopril, said first and second amounts in combination being effective to reduce blood pressure of a patient in need of treatment to within an acceptable value.

34. The composition of claim 33 wherein the first amount is within the range of 2.5-80 mg.

35. The composition of claim 33 wherein the second amount is within the range of 5-40 mg.

36. The composition of claim 35 wherein the first amount is within the range of 10-20 mg.

37. The composition of claim 35 wherein the second amount is within the range of 5-20 mg.

38. The composition of claim 33 wherein the lercanidipine and lisinopril content of said composition is selected from the following pairs (i) through (xxv): (i) said first amount is 2.5 mg per day and said second amount is 2.5 mg per day, (ii) said first amount is 2.5 mg per day and said second amount is 5 mg per day, (iii) said first amount is 2.5 mg per day and said second amount is 10 mg per day, (iv) said first amount is 2.5 mg per day and said second amount is 20 mg per day, (v) said first amount is 2.5 mg per day and said second amount is 40 mg per day, (vi) said first amount is 5 mg per day and said second amount is 2.5 mg per day, (vii) said first amount is 5 mg per day and said second amount is 5 mg per day, (viii) said first amount is 5 mg per day and said second amount is 10 mg per ay, (ix) said first amount is 5 mg per day and said second amount is 20 mg per day, (x) said first amount is 5 mg per day and said second amount is 40 mg per day, (xi) said first amount is 10 mg per day and said second amount is 2.5 mg per day, (xii) said first amount is 10 mg per day and said second amount is 5 mg per day, (xiii) said first amount is 10 mg per day and said second amount is 10 mg per day, (xiv) said first amount is 10 mg per day and said second amount is 20 mg per day, (xv) said first amount is 10 mg per day and said second amount is 40 mg per day, (xvi) said first amount is 20 mg per day and said second amount is 2.5 mg per day, (xvii) said first amount is 20 mg per day and said second amount is 5 mg per day, (xviii) said first amount is 20 mg per day and said second amount is 10 mg per day, (xix) said first amount is 20 mg per day and said second amount is 20 mg per day, (xx) said first amount is 20 mg per day and said second amount is 40 mg per day, (xxi) said first amount is 40 mg per day and said second amount is 2.5 mg per day, (xxii) said first amount is 40 mg per day and said second amount is 5 mg per day, (xxiii) said first amount is 40 mg per day and said second amount is 10 mg per day, (xxiv) said first amount is 40 mg per day and said second amount is 20 mg per day, (xxv) said first amount is 40 mg per day and said second amount is 40 mg per day, (xxvi) said first amount is 60 mg per day and said second amount is 2.5 mg per day, (xxvii) said first amount is 60 mg per day and said second amount is 5 mg per day, (xxviii) said first amount is 60 mg per day and said second amount is 10 mg per day, (xxix) said first amount is 60 mg per day and said second amount is 20 mg per day, (xxx) said first amount is 60 mg per day and said second amount is 40 mg per day, (xxxi) said first amount is 80 mg per day and said second amount is 2.5 mg per day, (xxxii) said first amount is 80 mg per day and said second amount is 5 mg per day, (xxxiii) said first amount is 80 mg per day and said second amount is 10 mg per day, (xxxiv) said first amount is 80 mg per day and said second amount is 20 mg per day, or (xxxv) said first amount is 80 mg per day and said second amount is 40 mg per day.

39. The method of claim 6, wherein said first amount of 60 or 80 mg is administered in a modified release formulation.

40. The method of claim 12, wherein said first amount of 60 or 80 mg is administered in a modified release formulation.

41. The method of claim 18, wherein said first amount of 60 or 80 mg is administered in a modified release formulation.

42. The method of claim 24, wherein said first amount of 60 or 80 mg is administered in a modified release formulation.

43. The method of claim 56, wherein said first amount of 60 or 80 mg is administered in a modified release formulation.

44. A method for treating hypertension in a patient in need thereof, comprising administering to said patient a sub-threshold amount of lercanidipine in combination with a sub-threshold amount of lisinopril, wherein said combination is effective to reduce blood pressure in said patient by at least a predetermined increment.

45. The method of claim 1 further comprising administering a third amount of a diuretic.

46. The method of claim 7 further comprising administering a third amount of a diuretic.

47. The method of claim 13 further comprising administering a third amount of a diuretic.

48. The method of claim 19 further comprising administering a third amount of a diuretic.

49. The method of claim 44 further comprising administering a third amount of a diuretic.

50. A pharmaceutical composition for treatment of hypertension comprising a first amount of lercanidipine, a second amount of lisinopril, and a third amount of a diuretic, said first, second and third amounts in combination being effective to reduce blood pressure of a patient in need of treatment to within an acceptable value.

51. The pharmaceutical composition of claim 50 wherein said diuretic is HCTZ.

52. A method for treating hypertension in a patient in need thereof, said method comprising administering to said patient a first amount of lercanidipine, a second amount of lisinopril, and third amount of a diuretic, to treat hypertension in said patient.

53. The method of claim 52 wherein said diuretic is HCTZ.

54. The method of claim 52 wherein said first, second, and third amounts together in combination are effective to reduce blood pressure in said patient by at least a predetermined increment; wherein each of said first amount, said second, and third amount administered individually is ineffective to produce a reduction in blood pressure in said patient or said reduction in blood pressure is less than said predetermined increment.

55. The method of claim 54 wherein said diuretic is HCTZ.
